(19) United States
(12) Patent Application Publication
(10) Pub. No.: US 2017/0191038 A1
(43) Pub. Date: Jul. 6, 2017

(54) TGFß SIGNALING INDEPENDENT NAÏVE INDUCED PLURIPOTENT STEM CELLS, METHODS OF MAKING AND USE

(71) Applicants: HONG GUAN Ltd., Beijing (CN); PEKING UNIVERSITY, Beijing (CN); BEIJING VITALSTAR BIOTECHNOLOGY CO., LTD., Beijing (CN)

(72) Inventors: Hongkui DENG, Beijing (CN); Riguo FANG, Beijing (CN); Kang LIU, Beijing (CN); Weifeng YANG, Beijing (CN)

(21) Appl. No.: 15/326,216

(22) PCT Filed: Sep. 18, 2015
(86) PCT No.: PCT/CN2015/089963

§ 371 Date: Jan. 13, 2017

(30) Foreign Application Priority Data
Sep. 26, 2014 (CN) 201410504189.8

(51) Int. Cl. C12N 5/074; A01K 67/027

(52) U.S. Cl. CPC C12N 5/0696; A01K 67/027; C12N 2501/727; C12N 2501/115; C12N 2501/20; C12N 2501/50; A01K 2267/025; A01K 2227/105; A01K 2207/12; C12N 2506/09

(57) ABSTRACT

Provided is a cocktail of factors for converting/reprogramming non-naïve pluripotent stem cells into TGFβ signaling-independent (TSI) naïve induced pluripotent stem cells (iPSCs). Also provided are methods for reprograming a non-naïve PSC into a TSI naïve iPSC by contacting the cell to be reprogrammed with effective amounts of compounds for a sufficient period of time to reprogram the cell into a TSI naïve iPSC.

### FIELD OF THE INVENTION

The invention is generally directed to TGF-β-signaling independent (TSI) naive induced pluripotent stem cells.

### BACKGROUND OF THE INVENTION

Studies in rodents have indicated that pluripotent stem cells (PSCs) can be classified into two distinct and stable pluripotent states: the naive and the primed pluripotent states (Nichols, et al. Cell Stem Cell, 4:487-492 (2009)). The naive state is represented by mouse embryonic stem cells (mESCs) derived from the inner cell mass (ICM) of preimplantation mouse blastocyst embryos (Evans, et al., Nature, 292:154-156 (1981); Brook, et al., Proc. Natl. Acad. Sci. USA, 94:5709-5712 (1997)), while the primed state corresponds to mouse epiblast stem cells (mEpiSCs) that have been established from mouse epiblasts, postimplantation (Tesar et al., Nature, 448:196-199 (2007); Brons et al., Nature, 448:191-195 2007). Moreover, naive and primed pluripotent stem cells possess different gene expression profiles and different signaling pathways to support their self-renewal. For instance, mESCs require LIF signaling or the combinatorial inhibition of extracellular regulated protein kinases (ERK) and glycogen synthase kinase-3 (GSK3), while mEpiSCs depend on basic fibroblast growth factor (bFGF) and transforming growth factor-β (TGF-β) signaling (Tesar et al., Nature, 448:196-199 (2007); Ying et al., Nature, 453:519-523 (2008)). In addition, female mESCs retain an active X chromosome status, but female mEpiESCs are kept in X-inactivation status. Furthermore, while primed PSCs form flattened colonies with a slow proliferation rate and are refractory to single-cell passaging, naive PSCs grow rapidly and can be propagated through single-cell passaging (Nichols, Cell Stem Cell, 4:487-49 (2009)). Most importantly, in contrast to primed pluripotent stem cells, naive PSCs possess no marked lineage commitment bias in vitro, and are capable of repopulating into the ICM of early blastocysts with high-grade chimerism in vivo (Bradley et al., Nature, 309:255-256 (1984); Nichols, Cell Stem Cell, 4:487-49 (2009)), making naïve PSCs important for creating chimeric animal models and studying mammalian gene function and early development.

Although the distinct naive and primed pluripotent states have been well established in rodents, conventional primate PSCs, such as human and monkey ESCs and induced pluripotent stem cells (iPSCs) more closely resemble postimplantation mouse EpiSCs in terms of gene expression profiles, signaling pathways required for proliferation, and intolerance to single cell passaging (Thomson et al., Proc. Natl. Acad. Sci., 92:7844-7848 (1995); Thomson et al., Science, 282:1145-1147 (1998); Takahashi et al., Cell, 131:861-872 (2007); Yu et al., Science, 318:1917-1920 (2007); Liu et al., Cell Stem Cell, 3:587-590 (2008)). Therefore, whether and how the naive pluripotent state in primates can be established remains an important question. Recent studies have reported deriving naive human PSCs in vitro by the conversion of primed PSCs or by direct reprogramming of somatic cells, such as the ectopic expression of LRH-1 and RARg or using small molecules (Smagghe et al., PLoS One, 8:e58601 (2013); Li et al., Cell Stem Cell, 4:16-19 (2009); Buecker et al., Cell Stem Cell, 6:535-546 (2010); Hanna et al., Proc. Natl. Acad. Sci. USA, 107:9222-9227 (2010); Wang et al., Proc. Natl. Acad. Sci. USA, 108:18283-18288 (2011)). Nevertheless, the absence of the complete set of rodent naive PSC characteristics indicates a need for a stable naïve pluripotent state in primates. Most recently, several reports have established distinct stable exogene-independent human naive pluripotent states in vitro (Gafni et al., Nature, 504:282-286 (2013); Chan et al., Cell Stem Cell, 13:663-675 (2013); Ware et al., Proc. Natl. Acad. Sci. USA, 111:4484-4489 (2014); Theunissen et al., Cell Stem Cell, 15(4):471-87 (2014)). Further, some studies have identified pluripotent stem cells which can be maintained in cell culture including expensive growth factors such as TGFβ. Since derivation of human naive PSCs requires different signaling pathways from the mouse, there is still a need for methods of generating naive PSCs from nonhuman primates in vitro for example, the rhesus monkey or naïve PSC using methods which eliminate the need for some growth factors.

It is therefore an object of the present invention to provide transforming growth factor signaling independent (TSI) naïve induced pluripotent stem cells.

It is also an object of the present invention to provide a method of converting non-naïve PSC into TSI naïve induced pluripotent stem cells.

It is still an object of the present invention to provide a method of maintaining TSI naïve induced pluripotent stem cells in the naïve state.

### SUMMARY OF THE INVENTION

Cocktails of factors/compounds have been identified which can be used to convert/reprogram non-naïve pluripotent stem cells into naïve pluripotent stem cells, herein after, cocktail. The cocktail is used to generate TGF-β-signaling independent (TSI) naive pluripotent stem cell, which are chemically induced into the naïve state (i.e., TSI naïve PSC) and to maintain the cells so generated in the naïve state. The cocktail of compounds include the following compounds, herein (chemical inducer of naive pluripotency (CINP)) in effective amounts which, in combination, reprogram a non-naïve PSC into a TSI naïve PSC: (1) a cytokine; (2) a glycogen synthase kinase (GSK) inhibitor; (3) an ERK 1/2 inhibitor; (4) a c-Jun N-terminal kinase/stress-activated protein kinase (JNK/MAPK) inhibitor (5) basic fibroblast growth factor and (6) a p38 mitogen-activated protein kinase inhibitor. In a preferred embodiment, the cytokine is Leukemia inhibitory factor (LIF) (“L); the GSK inhibitor is the aminopyrimidine, CHIR99021 (“C”) which has the chemical name [6-[[2-[[4-(2,4-Dichlorophenyl)-5-(5-methyl-1H-imidazol-2-yl)-2-pyrimidinyl]amino]ethyl]amino]-3-pyridinecarbonitrile]; ERK 1/2 inhibitor is PD0325901; JNK inhibitor is SP600125 (Anthra[1-9-cd]pyrazol-6(2H)-one); and the p38 inhibitor is SB203580 (4-[5-(4-Fluorophenyl)-2-[4-(methylsulfonyl)phenyl]-1H-imidazol-4-yl]pyridine). This cocktail of compounds in effective amounts can be used to reprogram non-naïve pluripotent stem cells into TSI naive PSC.

Also provided is a method of inducing/reprograming a non-naïve PSC into a naive PSC by reprogramming a donor cell using the cocktail of compounds disclosed herein. The cell to be reprogrammed (i.e., the donor cell) is contacted with the cocktail for a sufficient period of time to reprogram the cell into a TSI naive PSC. In some embodiments where the donor cell is not a pluripotent stem cell, the donor cell is first converted into a primed induced pluripotent stem cell (iPSC). In this embodiment, primed iPSC are cultured initially in the cocktail of compounds disclosed herein for a period between 4 and 14 days preferably between 7-10 days. In a preferred embodiment, the cell culture medium does not include a PKC inhibitor, a ROCK inhibitor, TGFβ, a NOTCH inhibitor, a TGFR inhibitor or an FGFR inhibitor. More preferably, the cocktail of compounds does not include TGFβ. The TSI naive PSC are isolated and maintained in the naïve state in pluripotent stem cell culture medium supplemented with the same cocktail of compounds used to generate them.

Also provided are TSI naive PSC. The TSI naïve PSC are so identified at least because of the ability to maintain of pluripotency independent of TGFβ1 signaling. A non-naïve reprogrammed PSC cell contacted with the cocktail of compounds as disclosed herein is identified as TSI naive PSC based on properties including: (i) morphologically (on the basis of formation of dome shaped colonies in culture), (ii) functionally, based on the following characteristics: (a) maintenance of pluripotency independent of TGFβ1 (as measured by fold change in the proportion of TRA-1-81-positive cells relative to controls in the presence of a TGFβ1 receptor inhibitor); (b) the ability of the cell to differentiate into tissues of the three embryonic germ layers; (c) upregulated expression of one or more naïve state related transcripts such as PRDM14, KLF5, ZFP42 (REX1), LIFR, TBX3, and NANOG, (d) upregulated expression of one or more markers for pluripotency such as TRA-1-60, TRA-1-81, and SSEA-4; (e) down regulation of one or markers for pluripotency such as SSEA-1; (f) the ability to form interspecies chimeras in vivo. The TSI naive PSC is different from a cell which has not been exposed to the cocktail disclosed herein in that it possesses at least one, preferably two, three, four or all of these properties, when compared to untreated cell. Upregulation or downregulation is determined by comparing the levels of the measured factor in the corresponding cell from which the TSI naive PSC was obtained.

The TSI naive PSC disclosed herein can be distinguished from human or mouse ESC or iPSC at least by the methods that are used to generate them i.e., by their origin. Where ESC are naturally occurring cells for example, TSI naive PSC on the other hand are not naturally occurring (as evidenced by possession of characteristics which are not found in corresponding naturally occurring ESC), when TSI naive PSC are obtained by treating non naïve PSC with a combination of small molecules, as described herein.

TSI naive PSC can be cultured or induced to differentiate into cells of a desired type. The TSI naive PSC and their progeny can be used in a number of applications, including but not limited to cell therapy, animal models and tissue engineering.

### BRIEF DESCRIPTION OF THE DRAWINGS

FIG. 1A is a bar graph showing colony numbers for naive iPSCs expanded under basal conversion conditions with the tested signaling modulators for 8 days after plating. Maintenance of pluripotency was measured by the proportion of TBX3 and TRA-1-81 double-positive colonies among total colonies. Negative control: 2i/LIF (Leukemia inhibitory factor)+bFGF (n=3 wells). All values are mean±SEM from 3-well replicates. FIGS. 1B and 1C show colony numbers for Rhesus monkey naïve iPSCs expanded under basal conversion conditions with the tested signaling modulators for 5 days after plating. Pluripotency maintenance was measured by the proportion of TBX3 or TRA-1-81-single positive colonies among the total colonies. Negative control: 2i/LIF+bFGF. All values are mean±s.e.m from 3-well replicates.

FIG. 2A shows quantitative RT-PCR analysis of the losing of episomal vectors integrated into the genome of established primed iPSC lines. Fibro-D6: fibroblasts after infection of episomal vectors for 6 days; EViPS-1, 2: 2 primed iPSC lines established by episomal vectors system. All values are mean±s.e.m from 3 independent experiments. FIG. 2B shows RT-PCR analysis of pluripotency marker gene expression in rhesus monkey directly converted into naïve iPSCs (DRN-1, DRN-2, DRN-3), female naïve iPSCs (FN-1, FN-2, FN-3) and episomal-induced naïve iPSCs (EVN-1, EVN-2, EVN-3). (ES-7.5: rhesus monkey ES line). FIG. 2C shows colony reformation after single-cell passaging of primed iPSCs and naive iPSCs. Pluripotency maintenance was measured by the number of TRA-1-81-positive colonies at day 5 after passaging (n=3 wells). p<0.0001 (Student's t test). All values are mean±SEM from 3-well replicates. FIG. 2D shows RT-PCR analysis of pluripotency gene expression in naive iPSCs. (N1, N2, N3: three independently established naive iPSC lines; ES-7.5: rhesus monkey ESC line).

FIGS. 3A and 3B show fold change in TRA-1-81+ domed colony numbers for naive iPSCs expanded under optimized conversion conditions (FIG. 3A). Primed iPSCs were expanded in the hESCs medium (FIG. 3B). The signaling modulators tested were as follows: 1 mM JAK inhibitor, 10 mM SB431542 (TGFβR inhibitor), 2 ng/ml TGF-b1, and 2 mM SU5402 (FGFR inhibitor). Pluripotency maintenance was measured by the fold change in the proportion of TRA-1-81-positive dome-shaped colonies relative to the controls (optimized conversion conditions for naive iPSCs) or TRA-1-81-positive colonies relative to the controls (hESCs medium for primed iPSCs). The different columns represent individual cell lines (n=3 different wells). N1, N2, N3: three naive iPSC lines; P1, P2, P3: three primed iPSC lines. All values are mean ±SEM from 3-well replicates. FIG. 3C shows show fold change in TRA-1-81+ colony numbers for Rhesus monkey naïve iPSCs expanded under optimized conversion conditions (2i/LIF+bFGF+SP600125+SB203580). Different concentrations of Ly294002 were tested as indicated for 5 days. Pluripotency maintenance was measured by the number of TRA-1-81 positive dome-shaped colonies. All values are mean±s.e.m from 3-well replicates. FIG. 3D shows show fold change in TRA-1-81+ domed colony numbers for Rhesus monkey naïve iPSCs expanded under optimized conversion conditions (2i/LIF+bFGF+SP600125+SB203580). In addition, 2 ng/ml TGF-β1 alone and 2 ng/ml TGF-β1 with 10 μM SB431542 (TGF-βR inhibitor) were tested. Pluripotency maintenance was measured by the fold change in TRA-1-81 positive dome-shaped colonies relative to the control 2i/LIF+bFGF+SP600125+SB203580). All values are mean±s.e.m from 3-well replicates. FIG. 3E shows qPCR and RT-PCR analysis of XIST expression (FN-1 and FN-2: female naive iPSC lines; FP-1 and FP-2: female primed iPSC lines; FF-1 and FF-2: female fibroblast cell lines; N1: a male naive iPSC line; MF: a male fibroblast cell line). The results of RT-PCR are shown relative to the average expression level of XIST in primed iPSCs (Female #1 and #2: two female cell sources; N: naive; P: primed; F: fibroblasts). All values are mean±SEM from three independent experiments. FIG. 3F shows clustering was performed on whole genomic expression profile (RNA-seq) of primed (P1, P2) and naive iPSCs (N1, N2, FN-1, FN-2) using hierarchical clustering. FIG. 3G shows Gene ontology (GO) analysis showing up and down regulated signaling pathway-related gene categories between monkey naïve and primed iPSCs. FIGS. 3H-J show quantitative PCR validation of typical pluripotency and lineage-specific marker gene expression in naive and primed iPSCs. All values are the mean±SD from three independent experiments. p<0.0001 (Student's t test).

FIGS. 4A and 4B show RT-PCR analysis of pluripotency marker gene expression in retroviral induced (OK-1, OK-2, OK-3) and episomal vectors-induced (EV-1, EV-2, EV-3) rhesus monkey primed iPSCs. “Endo” indicates endogenous gene expression. FIG. 4C is a bar graph showing quantitative RT-PCR analysis of the expression of all exogenous transcription factor genes. All values are mean±s.e.m from 3 independent experiments.

### DETAILED DESCRIPTION OF THE INVENTION

### I. Definitions

The term “chemically induced pluripotent stem cells” (ciPSCs) as used herein refers to pluripotent cells derived from a cell that is not pluripotent, i.e., a multipotent or differentiated cell, by contacting the non-pluripotent cell with chemical compounds, not by expression of one or more transfected genes.

“2i” as use herein refers to ESC culture medium with dual inhibition of glycogen synthase kinase-3 and mitogen-activated protein kinase signaling, for example, ESC culture medium supplemented with 2i (CHIR99021 and PD0325901).

The term “Induced pluripotent stem cell” (iPSC), as used herein, is a type of pluripotent stem cell artificially derived from a non-pluripotent cell. CiPSCs are iPSCs; however, they differ from some iPSCs in that they are not genetically engineered to confer pluripotency.

The term “isolated” or “purified” when referring to TSI naive PSC means chemically naïve induced naive pluripotent stem cells at least 10%, 20% 30%, 35%, 40%, 45%, 50%, 55%, 60%, 65%, 70%, 75%, 80%, 85%, 90%, 95%, or 99% free of contaminating cell types which are not naïve pluripotent cells. The isolated TSI naïve PSCs may also be substantially free of soluble, naturally occurring molecules.

“Media” and “culture medium” as used herein refers to the cell culture milieu. Media is typically an isotonic solution, and can be liquid, gelatinous, or semi-solid, for example, to provide a matrix for cell adhesion or support. Media, as used herein, can include the components for nutritional, chemical, and structural support necessary for culturing a cell.

The term “pluripotency” (or pluripotent), as used herein refers to a stem cell that has the potential to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system). A multipotent stem cell is less plastic and more differentiated, and can become one of several types of cells within a given organ. For example, multipotent blood stem cells can develop into red blood cell progenitors, white blood cells or platelet producing cells. Adult stem cells are multipotent stem cells. Adipose-derived stem cells are multipotent.

“Pluripotent cell is used herein interchangeably with, “pluripotent stem cell”.

The term “small molecule” refers to a molecule, such as an organic or organometallic compound, with a molecular weight of less than 2,000 Daltons, more preferably less than 1,500 Daltons, most preferably, less than 1,000 Daltons.

The term “TGFβ-signaling independent (TSI) naive PSC” as used herein refers to PSC to which the characteristic of being a “naïve” PSC is artificially conferred to a non-naïve PSC (donor cell) by culturing a non-naïve PSC cell in the cocktail of compounds disclosed herein, and where the conversion/reprogramming into a naïve PSC is independent of TGFβ signaling. The donor cell include non-PSC and PSCs whose ability to maintain pluripotency is TGFβ-signaling dependent. TGFβ signaling independence is determined by the ability of a naive PSC to maintain pluripotency when cultured for least 5 days, independent of TGFβ1 (as measured by fold change in the proportion of TRA-1-81-positive cells relative to controls in the presence of a TGFβ1 receptor inhibitor). An example of determining TGFβ-signaling independence of naïve PSC is provided under “Cell Culture” as discussed further below.

### II. Compositions

A cocktail of compounds for converting non-naïve pluripotent stem cells into TGF-β-signaling independent (TSI) naive PSC includes the compounds disclosed herein in effective amount to convert/reprogram the non-naïve PSC into a TGF-β-signaling independent (TSI) naive PSC, and to main the cells in the naïve state in culture. The cocktail of compounds preferably do not include a PKC inhibitor, a ROCK inhibitor, TGFβ, a NOTCH inhibitor, a TGFR inhibitor or an FGFR inhibitor. More preferably, the cocktail of compounds does not include TGFβ.

The compositions disclosed herein also include isolated TGF-β-signaling independent (TSI) naive PSC.

A. Cocktail of Compounds for Reprogramming Non-Naïve PSC into TGF-β-Signaling Independent (TSI) Naive PSC

The cocktail of compounds include a cytokine, small molecules and a protein factor such as basic fibroblast growth factor (bFGF). A most preferred cocktail includes 4 ng/ml bFGF, 10 ng/ml human LIF, CHIR99021 (3 μM) and PD0325901 (0.5 μM), and SP600125 (10 μM) and SB203580 (10 μM).

1. Cytokines

A preferred cytokine is human Leukemia inhibitory factor (LIF) (“L), an interleukin 6 class cytokine, used in a concentration range from 1-100 ng/ml, preferably from 1-50 and even more preferably, from 1 to 30 ng/ml. IL-6 is a prototypical four-helix bundle cytokine that is the founder member of the neuropoietins, a group of cytokines structurally related, that include IL-6, IL-11, IL-27, IL-31, leukemia inhibitory factor, oncostatin M, cardiotrophin-1, neuropoietin and cardiotrophin-like cytokine factor 1 (also known as new neurotrophin 1 and B cell stimulatory factor-3), and two viral analogs of IL-6. These members of the interleukin 6 family of cytokines can be used in the compositions disclosed herein, at equivalent concentrations disclosed for LIF.

2. Small Molecules

The chemical cocktail disclosed herein includes effective amounts of small molecules having a molecular weight of less than 2,000 Daltons, more preferably less than 1,500 Daltons, most preferably less than 1,000 Dalton, alone or in combination with proteins. The small molecules may have a molecular weight less than or equal to 900 Daltons or, less than or equal to 500 Daltons. Larger molecules can be used in chemically-induced reprogramming, preferably targeting the same pathway as the small molecules identified here.
(i) ERK 1/2 inhibitors

A preferred ERK 1/2 inhibitor is PD0325901 (N-[(2R)-2,3-Dihydroxypropoxy]-3,4-difluoro-2-[(2-fluoro-4-iodophenyl)amino]-benzamide) in a concentration range from 0.1 to 5 μM, preferably between 0.5 and 3, and even more preferably, between 1.5 and 1 μM For example, the cocktail can include PD0325901 in concentrations of 0.1, 0.2, 0.3, 0.4, 0.5, 0.6, 0.7, 0.8, 0.9, 1, 1.5, 2, 2.5, 3, 3.5, 4, 4.5, or 5 μM. However, other useful inhibitors include, but are not limited to, PD198306 (N-(Cyclopropylmethoxy)-3,4,5-trifluoro-2-[(4-iodo-2-methylphenyl)amino]-benzamide); SL 327 (α-[Amino[(4-aminophenyl)thio]methylene]-2-(trifluoromethyl)benzeneacetonitrile); and U0126 (1,4-Diamino-2,3-dicyano-1,4-bis[2-aminophenylthio]butadiene).
(ii) GSK inhibitors

The GSK inhibitor preferably inhibits GSK3 and preferably, is selective for GSK3. A suitable GSK inhibitor is the aminopyrimidine, CHIR99021, which is the glycogen synthase kinase 3 inhibitor. The CINP compositions include CHIR99021 in a concentration range from 0.01 to 20 μM, preferably between 1 and 3, and even more preferably, between 1.5 and 3 μM. For example, the CINP can include CHIR99021 in concentrations of 0.01, 0.05, 0.1, 1, 1.5, 2, 2.5, 3, 3.5, 4, 4.5, 5, 10, 20 μM. Concentrations that fall between these numbers are contemplated, as one of ordinary skill in the art can readily fine tune the effective amounts needed.

However, other GSK inhibitors are commercially available and are can be used in the compositions disclosed herein. Examples include, but are not limited to BIO-acetoxime; GSK 31 inhibitor XV; SB-216763; CHIR 99021 trihydrochloride, which is the hydrochloride salt of CHIR99021; GSK-3 Inhibitor IX [((2Z,3E)-6′-bromo-3-(hydroxyimino)-[2,3′-biindolinylidene]-2′-one]; GSK 3 IX [6-Bromoindirubin-3′-oxime]; GSK-3β Inhibitor XII [3-[[6-(3-Aminophenyl)-7H-pyrrolo[2,3-d]pyrimidin-4-yl]oxy]phenol]; GSK-3 Inhibitor XVI [6-(2-(4-(2,4-dichlorophenyl)-5-(4-methyl-1H-imidazol-2-yl)-pyrimidin-2-ylamino)ethyl-amino)-nicotinonitrile]; SB-415286 [3-[(3-chloro-4-hydroxyphenyl)amino]-4-(2-nitrophenyl)-1H-pyrrole-2,5-dione]; and Bio [(2′Z,3′E)-6-bromoindirubin-3′-oxime].

A non limiting list of useful compounds that can be included in the cocktail for reprogramming non naïve cells into TSI naïve PSC is provided in Table 1, including their structures.

TABLE 1
Useful chemical compounds

| | |
|---|---|
| CHIR99021 | 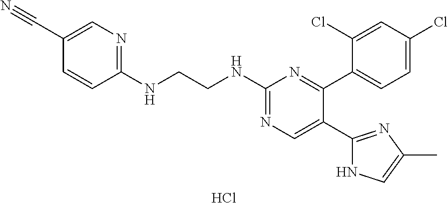 [SMILES: Cc1c[nH]c(-c2cnc(NCCNc3ccc(C#N)cn3)nc2-c2ccc(Cl)cc2Cl)n1 ; Cl] |
| CHIR98014 | 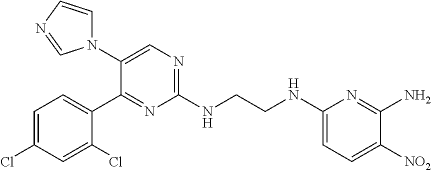 [SMILES: Nc1nc(NCCNc2ncc(-n3ccnc3)c(-c3ccc(Cl)cc3Cl)n2)ccc1[N+](=O)[O-]] |
| SB216763 | 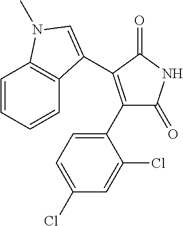 [SMILES: Cn1cc(C2=C(c3ccc(Cl)cc3Cl)C(=O)NC2=O)c2ccccc21] |
| TWS119 | 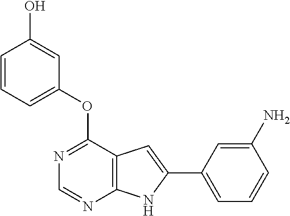 [SMILES: Nc1cccc(-c2cc3c(Oc4cccc(O)c4)ncnc3[nH]2)c1] |
| SB415286 | 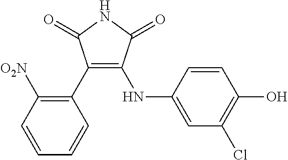 [SMILES: O=C1NC(=O)C(c2ccccc2[N+](=O)[O-])=C1Nc1ccc(O)c(Cl)c1] |
| LY2090314 | 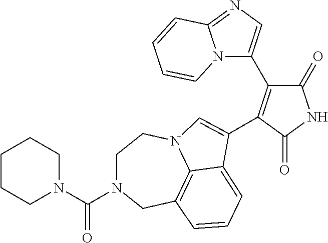 [SMILES: O=C1NC(=O)C(c2cnc3ccccn23)=C1c1cn2c3c(cccc13)CN(C(=O)N1CCCCC1)CC2] |
| Tideglusib | 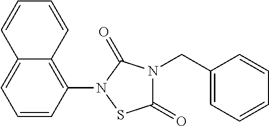 [SMILES: O=c1sn(-c2cccc3ccccc23)c(=O)n1Cc1ccccc1] |
| TDZD-8 | 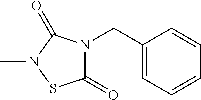 |
| CBM1078 | 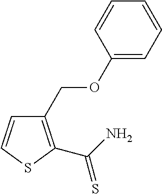 [SMILES: NC(=S)c1sccc1COc1ccccc1] |
| TD114-2 | 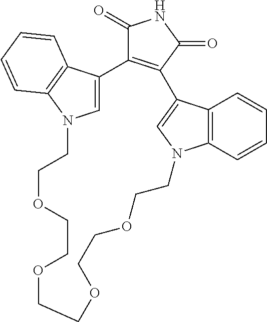 [SMILES: O=C1NC(=O)C2=C1c1cn(c3ccccc13)CCOCCOCCOCCOCCn1cc2c2ccccc21] |
| 3F8 | 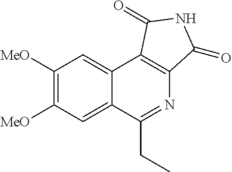 [SMILES: CCc1nc2c(c3cc(OC)c(OC)cc13)C(=O)NC2=O] |
| AR-A 014418 | 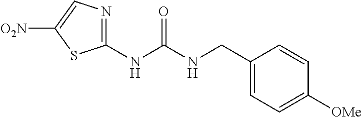 [SMILES: COc1ccc(CNC(=O)Nc2ncc([N+](=O)[O-])s2)cc1] |
| FRATide | Ser-Gln-Pro-Glu-Thr-Arg-Thr-Gly-Asp-Asp-Asp-Pro-His- |
| | Arg-Leu-Leu-Gln-Gln-Leu-Val-Leu-Ser-Gly-Asn-Leu-Ile- |
| | Lys-Glu-Ala-Val-Arg-Leu-His-Ser-Arg-Arg-Leu-Gln |
| Indirubin-3′- oxime | 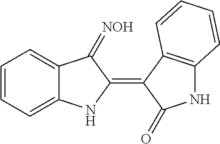 |
| L803 | Lys-Glu-Ala-Pro-Pro-Ala-Pro-Pro-Gln-pSer-Pro |
| Kenpaullone | 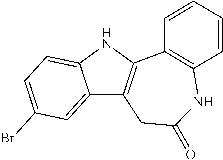 [SMILES: O=C1Cc2c([nH]c3ccc(Br)cc23)-c2ccccc2N1] |
| BIO | 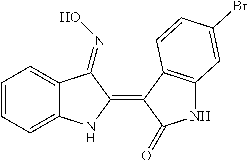 [SMILES: O=C1Nc2cc(Br)ccc2/C1=C1/Nc2ccccc2/C1=N\O] |

(iii) JNK inhibitors

A preferred JNK inhibitor is SP600125 (Anthra[1-9-cd]pyrazol-6(2H)-one) in a concentration range between 1 and 100 μM preferably from 1-50 and even more preferably, from 1 to 30 μM. For example, the cocktail of compositions can include 5, 10, 15, 20, 25 or 30 μM SP600125. Concentrations that fall between these numbers are contemplated, as one of ordinary skill in the art can readily fine tune the effective amounts needed.

Other useful JNK inhibitors include, but are not limited to BI 78D3 (4-(2,3-Dihydro-1,4-benzodioxin-6-yl)-2,4-dihydro-5-[(5-nitro-2-thiazolyl)thio]-3H-1,2,4-triazol-3-one); CEP 1347 ((9S,10R,12R)-5-16-Bis[(ethylthio)methyl]-2,3,9,10,11,12-hexahydro-10-hydroxy-9-methyl-1-oxo-9,12-epoxy-1H-diindolo[1,2,3-fg:3′,2′,1′-kl]pyrrolo[3,4-i][1,6]benzodiazocine-10-carboxylic acid methyl ester); and SU 3327 (−[(5-Nitro-2-thiazolyl)thio]-1,3,4thiadiazol-2-amine); AEG 3482 (6-Phenylimidazo[2,1-b]-1,3,4-thiadiazole-2-sulfonamide).
(iv) p38 MAPK inhibitors

A preferred p38 MAPK inhibitor is SB203580 (4-[5-(4-Fluorophenyl)-2-[4-(methylsulfonyl)phenyl]-1H-imidazol-4-yl]pyridine), used in a concentration range from between 1 and 100 μM preferably from 1-50 μM and even more preferably, from 1 to 30 μM. For example, the cocktail of compositions can include 5, 10, 15, 20, 25 or 30 μM SB203580. Other useful p38 MAPK inhibitors include, but are not limited to SB 203580 hydrochloride (4-[5-(4-Fluorophenyl)-2-[4-(methylsulphonyl)phenyl]-1H-imidazol-4-yl]pyridine hydrochloride); SB202190 (4-[4-(4-Fluorophenyl)-5-(4-pyridinyl)-1H-imidazol-2-yl]phenol); DBM 1285 dihydrochloride (N-Cyclopropyl-4-[4-(4-fluorophenyl)-2-(4-piperidinyl)-5-thiazolyl]-2-pyrimidinamine dihydrochloride); SB 239063 (trans-4-[4-(4-Fluorophenyl)-5-(2-methoxy-4-pyrimidinyl)-1H-imidazol-1-yl]cyclohexanol); SKF 86002 dihydrochloride (6-(4-Fluorophenyl)-2,3-dihydro-5-(4-pyridinyl)imidazo[2,1-b]thiazole dihydrochloride).

3. Protein Factors

Protein factors, such as recombinant basic fibroblast growth factor (bFGF), have been demonstrated to be effective in protocol for converting non-naïve PSC into the TSI naive PSC. bFGF can be used in a concentration range from 10 ng/mL-200 ng/mL, preferably at concentration of 10 ng/mL. Other factors which can be used include FGF1-18.

B. Cells to be Induced (Donor Cells)

TSI naive PSC are obtained by inducing/reprogramming pluripotent cells, or partially or completely differentiated cells obtained in some embodiments from a non-human primate such as a monkey, for example, Rhesus Monkey, chimpanzee, Gorillas, baboons, etc. In other embodiments, however, the cells to be induced/reprogramed are obtained from a human. Sources include bone marrow, fibroblasts, fetal tissue (e.g., fetal liver tissue), peripheral blood, umbilical cord blood, pancreas, skin or any organ or tissue.

In a preferred embodiment the TSI naive PSC are obtained from pluripotent cells, for example, embryonic stem cells or induced pluripotent stem cells (iPSCs). The iPSCs include cells obtained by genetic engineering and/or pure chemical reprograming. In other embodiments, TSI naive PSC are obtained from blactocyts.

Preferably, the iPSCs are obtained from chemically induced fibroblasts, adipose-derived stem cells, neural stem cells or cells from the intestinal epithelium. In some embodiment, TSI naive PSC are obtained from chemically induced neonatal (for example foreskin) or adult fibroblasts. However, iPSCs can be obtained from other cell types including but not limited to: multipotent stem cells, cells of hematological origin, cells of embryonic origin, skin derived cells, fibroblasts, adipose cells, epithelial cells, endothelial cells, mesenchymal cells, parenchymal cells, neurological cells, and connective tissue cells.

Pluripotent cells that can be used in the methods disclosed herein are known in the art and have been described, including methods of maintaining the cells in culture.

Donor cells may be isolated by disaggregating an appropriate organ or tissue which is to serve as the cell source using techniques known to those skilled in the art. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators.

C. TGF-β-Signaling Independent (TSI) Naive PSC

TSI naïve PSC can be so identified (i) morphologically, (ii) functionally, based on characteristics: (a) pluripotency maintenance independent of TGFβ1 (as measured by fold change in the proportion of TRA-1-81-positive cells relative to controls in the presence of a TGFβ1 receptor inhibitor); (b) the ability of the cell to differentiate into tissues of the three embryonic germ layers; (c) upregulated expression of one or more naïve state related transcripts such as PRDM14, KLFS, ZFP42 (REX1), LIFR, TBX3, and NANOG, (d) upregulated expression of one or more markers for pluripotency such as TRA-1-60, TRA-1-81, and SSEA-4; (e) down regulation of one or markers for pluripotency such as SSEA-1; (f) the ability to form interspecies chimeras in vivo.

TSI naive PSC form dome-shaped colonies. Accordingly, formation of dome-shaped colonies can be identified TSI naive PSC, following cell culture as exemplified herein. TSI naive PSC have the ability to differentiate into one or more cells/tissues from each of the three germ layers, the ectoderm, mesoderm and endoderm, using methods known in the art.

The ectoderm generates the outer layer of the embryo, and it forms from the embryo's epiblast. The ectoderm develops into the surface ectoderm, neural crest, and the neural tube. The surface ectoderm develops the epidermis, hair, nails, lens of the eye, sebaceous glands, cornea, tooth enamel, the epithelium of the mouth and nose. The neural crest of the ectoderm develops into: peripheral nervous system, adrenal medulla, melanocytes, facial cartilage. The neural tube of the ectoderm develops into: brain, spinal cord, posterior pituitary, motor neurons, and retina.

The endoderm consists at first of flattened cells, which subsequently become columnar. It forms the epithelial lining of the whole of the digestive tube except part of the mouth and pharynx and the terminal part of the rectum (which are lined by involutions of the ectoderm). It also forms the lining cells of all the glands which open into the digestive tube, including those of the liver and pancreas; the epithelium of the auditory tube and tympanic cavity; the trachea, bronchi, and air cells of the lungs; the urinary bladder and part of the urethra; and the follicle lining of the thyroid gland and thymus. The endoderm forms: the stomach, the colon, the liver, the pancreas, the urinary bladder, the epithelial parts of trachea, the lungs, the pharynx, the thyroid, the parathyroid, and the intestines.

The mesoderm forms connective tissue, muscle (smooth and striated), the lymphatic system, bone, serous membranes, cartilage, adipose tissue, circulatory system, dermis, genitourinary system, and notochord.

The TSI naive PSC can be additionally distinguished from an untreated corresponding in vitro cultured cell or other PSC, in their ability to maintain pluripotency independent of TGFβ1 (as measured by fold change in the proportion of TRA-1-81-positive cells relative to controls in the presence of a TGFβ1 receptor inhibitor). For example, in contrast to other PSC, TSI naive PSC can maintain pluripotency following at least 5 days of culture in the presence of TGFβ1 receptor inhibitor. An example of determining TGFβ-signaling independence of naïve PSC is provided under “Cell Culture”. Briefly, TSI naive PSC are cultured as disclosed therein for Rhesus monkey naïve iPSCs culture in optimized conversion medium. The optimized conversion medium can be supplemented with a TGFβ1 receptor inhibitor and TSI naive PSC in the supplemented medium, TRA-1-81-positive cells identified as described in the examples and compared to TSI naive PSC similarly cultured, without supplementation with TGFβ1 receptor inhibitor. Naïve cells as characterized as TSI naïve PSC based on a decrease in TRA-1-81-positive cells of less than 50%, preferably less than 40%, 30%, 20%, 10%, 5% or less, when compared to the control in this assay, i.e., if the decrease in TRA-1-81-positive cells is more than 2 fold, the naïve PSC is not characterized as a TSI naïve PSC as defined herein. Alternatively, or additionally, the TSI naive PSC can be distinguished from other PSC in their inability to maintain pluripotency in the presence of a selective inhibitor of Rho-associated, coiled-coil containing protein kinase (ROCK), for example, Y27632 [(+)-(R)-trans-4-(1-aminoethyl)-N-(4-pyridyl)cyclohexanecarboxamide+++dihydrochloride)] (10 μM) and a protein kinase C (PKC) inhibitor, for example, G06983 [3-[1-[3-(Dimethylamino)propyl]-5-methoxy-1H-indol-3-yl]-4-(1H-indol-3-yl)-1H-pyrrole-2,5-dione] (5 μM), as measured by pronounced differentiation and reduced TRA-1-81 expression in colonies obtained from treated cells when compared to untreated controls.

### III. Methods of Making

A. Reprogramming Non PSC into TSI Naive PSC

TSI naive PSC are produced by contacting cells to be induced/reprogrammed (herein donor cells) with culture media containing the cocktail of compounds disclosed herein for a sufficient period of time to result in reprograming the cells into TSI naive PSC.

A donor cell is contacted with the cocktail disclosed herein in an amount effective to induce and/or enhance reprograming of the cell into TSI naive PSC. One of skill in the art can readily determine the concentrations of the compounds disclosed herein required to provide complete reprograming, by using methods outlined in the examples below, or other methods known in the art. In a preferred embodiment, the donor is a pluripotent stem cell, for example as embryonic stem cells or induced pluripotent stem cells (iPSCs). The iPSCs include cells obtained by genetic engineering and/or pure chemical reprograming. In other embodiments, TSI naive PSC are obtained from blactocyts.

In embodiments where the donor cell is not a pluripotent stem cell, for example, a fibroblast cell, the donor cell is can be converted into a primed induced pluripotent stem cell (iPSC) before reprogramming into a TSI naïve PSC. Alternatively, the cells can be converted directly into TSI naïve PSC. Methods for converting non-pluripotent stem cells into induced pluripotent stem cells are known in the art, and are described for example, in Liu, et al., Cell Stem Cell, 3:587-590 (2008); Zhao, et al., Cell Stem Cell, 3:475-479 (2008), Okita, et al., Nat. Methods, 8:409-412 (2011)). iPSC are maintained in culture media for iPSC (as previously described) following induction into pluripotency, for a period of time between 20-40 days, preferably, between 25-35 days before reprogramming into TSI naïve PSC (Liu, et al., Cell Stem Cell, 3:587-590 (2008); Zhao, et al., Cell Stem Cell, 3:475-479 (2008), Okita, et al., Nat. Methods, 8:409-412 (2011)). For example, following transfection of fibroblasts with retroviral factors containing reprogramming factors (OCT4, KL4) as described in Liu, et al., Cell Stem Cell, 3:587-590 (2008), iPSC so generated are cultured in stem cell culture media supplemented with small molecules as described in the examples, with the medium being changed every two days. iPSC colonies are selected around day 20-40, preferably, around day 25-35, for reprogramming into TSI naïve PSC and maintained on feeder cells in the culture conditions for iPS. To reprogram iPSC into TSI naïve PSC, primed iPSC are cultured initially in the cocktail of compounds disclosed herein for a period between 4 and 14 days preferably between 7-10 days.

For direct conversion on non-pluripotent stem cells for example, fibroblasts into TSI naive PSC, cells are infected with retroviral vector containing reprogramming factors as described for example Liu, et al., Cell Stem Cell, 3:587-590 (2008) and maintained in basal medium (DF12, 20% KSR, basic Fibroblast Growth Factor) for a period of time ranging from 15-35 days, preferably, between 15 and 20 days, more preferably, for 20 days. The cell culture medium is then exchanged for cell culture medium containing the cocktail of factors for reprogramming non-naïve PSC into TSI naïve PSC, for a period between 7-10 days.

In a preferred embodiment, the cell culture medium for reprogramming cells into TSI naïve PSC does not include a PKC inhibitor, a ROCK inhibitor, TGFβ, a NOTCH inhibitor, a TGFR inhibitor or an FGFR inhibitor. More preferably, the cocktail of compounds does not include TGFβ. The TSI naive PSC are isolated and maintained in the naïve state in suitable stem cell culture medium including the same cocktail of compounds used to generate them.

Resultant cells are identified as TSI naive PSC (i) morphologically, (ii) functionally, based on characteristics: (a) pluripotency maintenance independent of TGFβ1 (as measured by fold change in the proportion of TRA-1-81-positive cells relative to controls in the presence of a TGFβ1 receptor inhibitor), determined for example, as disclosed herein; (b) the ability of the cell to differentiate into tissues of the three embryonic germ layers; (c) upregulated expression of one or more naïve state related transcripts such as PRDM14, KLF5, ZFP42 (REX1), LIFR, TBX3, and NANOG, (d) upregulated expression of one or more markers for pluripotency such as TRA-1-60, TRA-1-81, and SSEA-4; (e) down regulation of one or markers for pluripotency such as SSEA-1; (f) the ability to form interspecies chimeras in vivo.

B. Isolation of TSI Naive PSC

A substantially purified population of TSI naive PSC can be obtained, for example, by extraction (e.g., via density gradient centrifugation and/or flow cytometry) from a culture source. Purity can be measured by any appropriate method. The pluripotent cells can be 99%-100% purified by, for example, flow cytometry (e.g., FACS analysis). TSI naive PSC can be isolated by, for example, utilizing molecules (e.g., antibodies, antibody derivatives, ligands or Fc-peptide fusion molecules) that bind to a marker or a combination of markers on the induced pluripotent stem cells and thereby positively selecting cells that bind the molecule (i.e., a positive selection). Other examples of positive selection methods include methods of preferentially promoting the growth of a desired cell type in a mixed population of desired and undesired cell types. Alternatively, by using molecules that bind to markers that are not present on the desired cell type, but that are present on an undesired cell type, the undesired cells containing such markers can be removed from the desired cells (i.e., a negative selection). Other negative selection methods include preferentially killing or inhibiting the growth of an undesired cell type in a mixed population of desired and undesired cell types. Accordingly, by using negative selection, positive selection, or a combination thereof, an enriched population of stem cell can be made.

Procedures for separation may include magnetic separation, using antibody-coated magnetic beads, affinity chromatography, cytotoxic agents joined to a monoclonal antibody, or such agents used in conjunction with a monoclonal antibody, e.g., complement and cytotoxins, and “panning” with antibody attached to a solid matrix (e.g., plate), or other convenient technique. Techniques providing accurate separation include fluorescence activated cell sorters, which can have varying degrees of sophistication, e.g., a plurality of color channels, low angle and obtuse light scattering detecting channels, and impedance channels. Antibodies may be conjugated with markers, such as magnetic beads, which allow for direct separation, biotin, which can be removed with avidin or streptavidin bound to a support, or fluorochromes, which can be used with fluorescence activated cell sorter, to allow for ease of separation of the particular cell type. Any technique may be employed which is not unduly detrimental to the viability of the induced pluripotent stem cells. In one embodiment, the cells are incubated with an antibody against a marker (e.g., a TRA-1-81 antibody) and the cells that stain positive for the marker are manually selected and subcultured.

Combinations of enrichment methods may be used to improve the time or efficiency of purification or enrichment. For example, after an enrichment step to remove cells having markers that are not indicative of the cell type of interest, the cells may be further separated or enriched by a fluorescence activated cell sorter (FACS) or other methodology having high specificity. Multi-color analyses may be employed with a FACS. The cells may be separated on the basis of the level of staining for a particular antigen or lack thereof. Fluorochromes may be used to label antibodies specific for a particular antigen. Such fluorochromes include phycobiliproteins, e.g., phycoerythrin and allophycocyanins, fluorescein, and Texas red.

C. Culture and Preservation of TSI Naive PSC (and Their Progeny)

The TSI naive PSC can be expanded in culture and stored for later retrieval and use. Once a culture of cells or a mixed culture of stem cells is established, the population of cells is mitotically expanded in vitro by passage to fresh medium as cell density dictates under conditions conducive to cell proliferation, with or without tissue formation. Such culturing methods can include, for example, passaging the cells in culture medium lacking particular growth factors that induce differentiation (e.g., IGF, EGF, FGF, VEGF, and/or other growth factor). Cultured cells can be transferred to fresh medium when sufficient cell density is reached.

In a preferred embodiment, cell culture medium for maintaining TSI naive PSC is for example, N2B27 medium, supplemented with the cocktail of compounds disclosed herein, at the same concentrations used to induce naïve pluripotency i.e., the cocktail of compounds disclosed herein are used to reprogram a non naïve pluripotent cell into a TSI naive PSC, and to maintain the cells in the naïve state. For example, the cell culture medium for maintaining TSI naïve PSC can b N2B27 medium (without BSA), N2B27 medium (without BSA) supplemented with 5% KSR (Knockout serum replacement). Other basal media can also be used, for example, DF12 medium supplemented with 20% KSR. These basal media are supplemented with the cocktail of compounds as disclosed above. According to some embodiments of the invention, the cell culture media including effective amounts of the cocktail of compounds disclosed herein can maintain TSI naive PSC the undifferentiated and naive state 2 to over 100 passages in culture. For example, the cocktail can maintain TSI naive PSC in the undifferentiated and naïve state for 2, passages. 3, 4, 5, 6, 7, 8, 9 or 10 passaged in culture, preferably, for more than 10 passages, for example for about 20 passages in culture, e.g., for at least about 25, about 30, about 35, about 40, about 45, about 50, about 55, about 60, about 65, about 70, about 75 and about 80 passages while in culture. In a preferred embodiment, the TSI naive PSC maintain a normal karyotype during the 2, 3, 4, 5, 6, 7, 8, 9, 10, more than 10, for example, about 20 passages in culture, e.g., for at least about 25, about 30, about 35, about 40, about 45, about 50, about 55, about 60, about 65, about 70, about 75 and about 80 passages while in culture. As exemplified herein, the disclosed cocktail of compounds can maintain single cell passaged TSI naïve PSC in a normal karyotype for at least 8 months in culture.

Cells can be cryopreserved for storage according to known methods, such as those described in Doyle et al., (eds.), 1995, Cell & Tissue Culture: Laboratory Procedures, John Wiley & Sons, Chichester. For example, cells may be suspended in a “freeze medium” such as culture medium containing 15-20% fetal bovine serum (FBS) and 10% dimethylsulfoxide (DMSO), with or without 5-10% glycerol, at a density, for example, of about 4-10×10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years.

### IV. Methods of Using

Identification of a readily available source of stem cells that can give rise to a desired cell type or morphology is important for therapeutic treatments, tissue engineering and research. The availability of stem cells would be extremely useful in transplantation, tissue engineering, regulation of angiogenesis, vasculogenesis, organ regeneration, animal models, cell replacement or cell therapies as well as the prevention of diseases, etc. Such stem cells can also be used to introduce a gene into a subject as part of a gene therapy regimen.

A. Providing Differentiated Somatic Cells (Re-Differentiated Cells)

Once established, a culture of stem cells may be used to produce progeny cells, for example, fibroblasts capable of producing new tissue. The

TSI naive PSC can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells, blood cells, retinal cells (and other cells involved in sensory perception, such as those that form hair cells in the ear or taste buds on the tongue), and nervous tissue including nerves.

In one embodiment, the TSI naive PSC are induced to differentiate into cells of ectodermal origin by exposing the cells to an “ectodermal differentiating” media. In another embodiment the TSI naive PSC are induced to differentiate into cells of mesodermal origin by exposing the cells to “mesodermal differentiating media”. In still another embodiment, the TSI naive PSC are induced to differentiate into cells of endodermal origin by exposing the cells to “endodermal media”. Components of “endodermal”, “mesodermal” and “ectodermal” media are known to one of skill in the art. Known cell surface markers can be used to verify that the cells are indeed differentiating into cells of the lineage of the corresponding cell culture medium. The most commonly accepted markers to confirm differentiation of the three germ layers are the expression of alpha fetal protein for endodermal cells, alpha smooth muscle actin for mesoderm, and Beta-III tubulin for ectoderm, all of which are normally expressed very early in the development of these tissues.

Differentiation of stem cells to fibroblasts or other cell types, followed by the production of tissue therefrom, can be triggered by specific exogenous growth factors or by changing the culture conditions (e.g., the density) of a stem cell culture. Methods for inducing differentiation of cells into a cell of a desired cell type are known in the art. For example, TSI naive PSC can be induced to differentiate by adding a substance (e.g., a growth factor, enzyme, hormone, or other signaling molecule) to the cell's environment. Examples of factors that can be used to induce differentiation include erythropoietin, colony stimulating factors, e.g., GM-CSF, G-CSF, or M-CSF, interleukins, e.g., IL-1, -2, -3, -4, -5, -6, -7, -8, Leukemia Inhibitory Factory (LIF), or Steel Factor (Stl), coculture with tissue committed cells, or other lineage committed cells types to induce the stem cells into becoming committed to a particular lineage.

The redifferentiated cells can be can be expanded in culture and stored for later retrieval and use.

B. Cell Therapy

Therapeutic uses of TSI naïve PSC include transplanting the induced pluripotent stem cells, stem cell populations, or progeny thereof into individuals to treat a variety of pathological states including diseases and disorders resulting from cancers, wounds, neoplasms, injury, viral infections, diabetes and the like. Treatment may entail the use of the cells to produce new tissue, and the use of the tissue thus produced, according to any method presently known in the art. The cells may be implanted, injected or otherwise administered directly to the site of tissue damage so that they will produce new tissue in vivo. In one embodiment, administration includes the administration of genetically modified TSI naive PSC or their progeny.

In a preferred embodiment, the TSI naive PSC are obtained from autologous cells i.e., the donor cells are autologous. However, the cells can be obtained from heterologous cells. In one embodiment, the donor cells are obtained from a donor genetically related to the recipient. In another embodiment, donor cells are obtained from a donor genetically un-related to the recipient.

If the TSI naive PSC are derived from a heterologous (non-autologous/allogenic) source compared to the recipient subject, concomitant immunosuppression therapy is typically administered, e.g., administration of the immunosuppressive agent cyclosporine or FK506. However, due to the immature state of the human induced pluripotent stem cells such immunosuppressive therapy may not be required. Accordingly, in one embodiment, the human induced pluripotent stem cells can be administered to a recipient in the absence of immunomodulatory (e.g., immunsuppressive) therapy. Alternatively, the cells can be encapsulated in a membrane, which permits exchange of fluids but prevents cell/cell contact. Transplantation of microencapsulated cells is known in the art, e.g., Balladur et al., Surgery, 117:189-94, 1995; and Dixit et al., Cell Transplantation, 1:275-79 (1992).
(i) Diabetes

Diabetes mellitus (DM) is a group of metabolic diseases where the subject has high blood sugar, either because the pancreas does not produce enough insulin, or, because cells do not respond to insulin that is produced. A promising replacement for insulin therapy is provision of islet cells to the patient in need of insulin. Shapiro et al., N Engl J Med., 343(4):230-8 (2000) have demonstrated that transplantation of beta cells/islets provides therapy for patients with diabetes. Although numerous insulin types are commercially available, these formulations are provided as injectables. The human induced pluripotent stem cells provide an alternative source of islet cells to prevent or treat diabetes. For example, induced pluripotent stem cells can be isolated and differentiated to a pancreatic cell type and delivered to a subject. Alternatively, the induced pluripotent stem cells can be delivered to the pancreas of the subject and differentiated to islet cells in vivo. Accordingly, the cells are useful for transplantation in order to prevent or treat the occurrence of diabetes. Methods for reducing inflammation after cytokine exposure without affecting the viability and potency of pancreatic islet cells are disclosed for example in U.S. Pat. No. 8,637,494 to Naziruddin, et al.
(ii) Neurodegenerative Disorders

Neurodegenerative disorders are characterized by conditions involving the deterioration of neurons as a result of disease, hereditary conditions or injury, such as traumatic or ischemic spinal cord or brain injury. Neurodegenerative conditions include any disease or disorder or symptoms or causes or effects thereof involving the damage or deterioration of neurons. Neurodegenerative conditions can include, but are not limited to, Alexander Disease, Alper's Disease, Alzheimer Disease, Amyotrophic Lateral Sclerosis, Ataxia Telangiectasia, Canavan Disease, Cockayne Syndrome, Corticobasal Degeneration, Creutzfeldt-Jakob Disease, Huntington Disease, Kennedy's Disease, Krabbe Disease, Lewy Body Dementia, Machado-Joseph Disease, Multiple Sclerosis, Parkinson Disease, Pelizaeus-Merzbacher Disease, Niemann-Pick's Disease, Primary Lateral Sclerosis, Refsum's Disease, Sandhoff Disease, Schilder's Disease, Steele-Richardson-Olszewski Disease, Tabes Dorsalis or any other condition associated with damaged neurons. Other neurodegenerative conditions can include or be caused by traumatic spinal cord injury, ischemic spinal cord injury, stroke, traumatic brain injury, and hereditary conditions.

In particular, the disclosed methods include transplanting into a subject in need thereof NSCs, neural progenitors, or neural precursors that have been expanded in vitro such that the cells can ameliorate the neurodegenerative condition. Transplantation of the expanded neural stem cells can be used to improve ambulatory function in a subject suffering from various forms of myelopathy with symptoms of spasticity, rigidity, seizures, paralysis or any other hyperactivity of muscles. Methods for expanding and transplanting neural cells and neural progenitor cells for the treatment of different neurodegenerative conditions is disclosed for example, in U.S. Pat. No. 8,236,299 to Johe, et. al.
(iv) Cancer Therapy

Therapeutic uses of the TSI naive PSC and their progeny include transplanting the induced pluripotent stem cells, stem cell populations, or progeny thereof into individuals to treat and/or ameliorate the symptoms associated with cancer. For example, in one embodiment, the TSI naive PSC can be administered to cancer patients who have undergone chemotherapy that has killed, reduced, or damaged cells of a subject. In a typical stem cell transplant for cancer, very high doses of chemotherapy are used, often along with radiation therapy, to try to destroy all the cancer cells. This treatment also kills the stem cells in the bone marrow. Soon after treatment, stem cells are given to replace those that were destroyed.

In another embodiment, the TSI naive PSC can be transfected or transformed (in addition to the de-differentiation factors) with at least one additional therapeutic factor. For example, once TSI naive PSC are isolated, the cells may be transformed with a polynucleotide encoding a therapeutic polypeptide and then implanted or administered to a subject, or may be differentiated to a desired cell type and implanted and delivered to the subject. Under such conditions the polynucleotide is expressed within the subject for delivery of the polypeptide product.
(v) Tissue Engineering

TSI naive PSC and their progeny can be used to make tissue engineered constructions, using methods known in the art. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. The most commonly used biomaterial scaffolds for transplantation of stem cells are reviewed in the most commonly used biomaterial scaffolds for transplantation of stem cells is reviewed in Willerth, S. M. and Sakiyama-Elbert, S. E., Combining stem cells and biomaterial scaffolds for constructing tissues and cell delivery (Jul. 9, 2008), StemBook, ed. The Stem Cell Research Community, StemBook. Tissue engineering technology frequently involves selection of an appropriate culture substrate to sustain and promote tissue growth. In general, these substrates should be three-dimensional and should be processable to form scaffolds of a desired shape for the tissue of interest.

U.S. Pat. No. 6,962,814 generally discloses method for producing tissue engineered constructs and engineered native tissue. With respect to specific examples, U.S. Pat. No. 7,914,579 to Vacanti, et al., discloses tissue engineered ligaments and tendons. U.S. Pat. No. 5,716,404 discloses methods and compositions for reconstruction or augmentation of breast tissue using dissociated muscle cells implanted in combination with a polymeric matrix. U.S. Pat. No. 8,728,495 discloses repair of cartilage using autologous dermal fibroblasts. U.S. Published application No. 20090029322 by Duailibi, et al., discloses the use of stem cells to form dental tissue for use in making tooth substitute. U.S. Published application No. 2006/0019326 discloses cell-seed tissue-engineered polymers for treatment of intracranial aneurysms. U.S. Published application No. 2007/0059293 by Atala discloses the tissue-engineered constructs (and method for making such constructs) that can be used to replace damaged organs for example kidney, heart, liver, spleen, pancreas, bladder, ureter and urethra.
(vi) Cells produced from TSI Naive PSC (Progeny)

The TSI naive PSC can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells (e.g., alpha cells, delta cells, PP cells, and beta cells), blood cells (e.g., leukocytes, erythrocytes, macrophages, and lymphocytes), retinal cells (and other cells involved in sensory perception, such as those that form hair cells in the ear or taste buds on the tongue), and nervous tissue including nerves.
(vii) Therapeutic Compositions

The TSI naive PSC can be formulated for administration, delivery or contacting with a subject, tissue or cell to promote de-differentiation in vivo or in vitro/ex vivo. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

The induced pluripotent cells can be administered to a patient by way of a composition that includes a population of TSI naive PSC or TSI naive PSC progeny alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer.

In other embodiments, the cells are provided with or incorporated onto or into a support structure. Support structures may be meshes, solid supports, scaffolds, tubes, porous structures, and/or a hydrogel. The support structures may be biodegradable or non-biodegradable, in whole or in part. The support may be formed of a natural or synthetic polymer, metal such as titanium, bone or hydroxyapatite, or a ceramic. Natural polymers include collagen, hyaluronic acid, polysaccharides, and glycosaminoglycans. Synthetic polymers include polyhydroxyacids such as polylactic acid, polyglycolic acid, and copolymers thereof, polyhydroxyalkanoates such as polyhydroxybutyrate, polyorthoesters, polyanhydrides, polyurethanes, polycarbonates, and polyesters. These may be in for the form of implants, tubes, meshes, or hydrogels.

Solid Supports

The support structure may be a loose woven or non-woven mesh, where the cells are seeded in and onto the mesh. The structure may include solid structural supports. The support may be a tube, for example, a neural tube for regrowth of neural axons. The support may be a stent or valve. The support may be a joint prosthetic such as a knee or hip, or part thereof, that has a porous interface allowing ingrowth of cells and/or seeding of cells into the porous structure. Many other types of support structures are also possible. For example, the support structure can be formed from sponges, foams, corals, or biocompatible inorganic structures having internal pores, or mesh sheets of interwoven polymer fibers. These support structures can be prepared using known methods.

The support structure may be a permeable structure having pore-like cavities or interstices that shape and support the hydrogel-cell mixture. For example, the support structure can be a porous polymer mesh, a natural or synthetic sponge, or a support structure formed of metal or a material such as bone or hydroxyapatite. The porosity of the support structure should be such that nutrients can diffuse into the structure, thereby effectively reaching the cells inside, and waste products produced by the cells can diffuse out of the structure

The support structure can be shaped to conform to the space in which new tissue is desired. For example, the support structure can be shaped to conform to the shape of an area of the skin that has been burned or the portion of cartilage or bone that has been lost. Depending on the material from which it is made, the support structure can be shaped by cutting, molding, casting, or any other method that produces a desired shape. The support can be shaped either before or after the support structure is seeded with cells or is filled with a hydrogel-cell mixture, as described below.

An example of a suitable polymer is polyglactin, which is a 90:10 copolymer of glycolide and lactide, and is manufactured as VICRYL™ braided absorbable suture (Ethicon Co., Somerville, N.J.). Polymer fibers (such as VICRYL™), can be woven or compressed into a felt-like polymer sheet, which can then be cut into any desired shape. Alternatively, the polymer fibers can be compressed together in a mold that casts them into the shape desired for the support structure. In some cases, additional polymer can be added to the polymer fibers as they are molded to revise or impart additional structure to the fiber mesh. For example, a polylactic acid solution can be added to this sheet of polyglycolic fiber mesh, and the combination can be molded together to form a porous support structure. The polylactic acid binds the crosslinks of the polyglycolic acid fibers, thereby coating these individual fibers and fixing the shape of the molded fibers. The polylactic acid also fills in the spaces between the fibers. Thus, porosity can be varied according to the amount of polylactic acid introduced into the support. The pressure required to mold the fiber mesh into a desirable shape can be quite moderate. All that is required is that the fibers are held in place long enough for the binding and coating action of polylactic acid to take effect.

Alternatively, or in addition, the support structure can include other types of polymer fibers or polymer structures produced by techniques known in the art. For example, thin polymer films can be obtained by evaporating solvent from a polymer solution. These films can be cast into a desired shaped if the polymer solution is evaporated from a mold having the relief pattern of the desired shape. Polymer gels can also be molded into thin, permeable polymer structures using compression molding techniques known in the art.

Hydrogels

In another embodiment, the cells are mixed with a hydrogel to form a cell-hydrogel mixture. Hydrogels may be administered by injection or catheter, or at the time of implantation of other support structures. Crosslinking may occur prior to, during, or after administration.

D. Animal Models and Organ Regeneration

Isolated TSI naïve PSC can be used to generate animal models incorporating TSI naïve PSC from a desired species (donor) into a second animal (recipient) of the same or different species. The donor animal can be a mammal such as a human, mouse, rat, pig, cattle, sheep, goat, horse, dog, chimpanzee, gorilla, orangutan, monkey, marmoset, etc. In some preferred embodiments, the donor mammal is a human and the recipient mammal is non human, used to provide an animal model. In other embodiments, the donor and recipient animals are size matched. The recipient may be any animal other than human, such as pig, rat, mouse, cattle, sheep, goat, horse, dog, chimpanzee, gorilla, orangutan, monkey, marmoset, and bonobo. The TSI naive PSC can be used for organ regeneration in a mammal, which is not a human; TSI naive PSC can be used to produce a desired organ in the mammal where the mammal has an abnormality associated with a lack of development of that organ in a development stage.

The method includes transplanting TSI naive PSC into a blastocyst stage fertilized egg of the recipient non-human mammal; developing the fertilized egg in a womb of a non-human surrogate parent mammal to obtain a litter, and obtaining the organ from the litter, using methods known in the art. Examples of organs that can be produced include, but are not limited to, solid organ with a fixed shape, such as kidney, heart, pancreas, cerebellum, lung, thyroid gland, hair, and thymus. The recipient embryo may be from any animal other than human, such as pig, rat, mouse, cattle, sheep, goat, horse, dog, chimpanzee, gorilla, orangutan, monkey, marmoset, etc.

Methods for generating humanized mouse models are known in the art (U.S. Publication No. 20110258715) and reviewed for example in Ito, et al., Cellular & Molecular Immunology, 9:208-214 (2012). Examples of recipient embryos having an abnormality associated with the development of an organ of interest, and which can be used to regenerated that organ include, Sall1 knockout animal having an abnormality associated with a lack of development of a kidney in the development stage (Nishinakamura, et al., Development, 128:3105-3115 (2001); a Pdx1 knockout animal having an abnormality associated with a lack of development of a pancreas in the development stage (Offield, et al., Development, 122: 983-995 (1996); a Wnt-1 (int-1) knockout animal having an abnormality associated with a lack of development of a cerebellum in the development stage (McMahon, et al., Cell, 62:1073-1085, (1990); a T/ebp knockout animal having an abnormality associated with a lack of development of a lung and a thyroid gland in the development stage (Kimura, et al., Genes and Development, 10:60-69, 1996); or a dominant negative-type transgenic mutant animal model which overexpresses the deficiency of an intracellular domain of fibroblast growth factor (FGF) receptor (FGFR), and which causes deficiencies of multiple organs such as kidney and lung (Celli, et al., EMBO J., 17:1642-655, (1998)), can be used. Alternatively, nude mice can be used to produce of hair or thymus. A “founder” animal described U.S. Publication No. 20110258715 may also be used.

### V. Kits

Kits are provided which include the chemical cocktails disclosed herein. The chemical cocktails are as described above. These may be in a form having defined concentrations to facilitate addition to cell culture media to produce a desired concentration. The kit may include directions providing desired concentration ranges and times of administration based on the donor cell types. The kit may also include cell culture media which is pre-mixed with the chemical cocktail for culture of donor cells to induce naive pluripotency.

The present invention will be further understood by reference to the following non-limiting examples.

### EXAMPLES

Experimental Procedures

Generation of Rhesus Monkey Naive iPSCs from Primed iPSCs

The adult rhesus macaques (Macaca mulatta, #9, #2, #11, and #12) used in this study were housed in individual cages. All animal procedures were approved by the Laboratory Animal Center of the Chinese Academy of Military Medical Science, and the use of rhesus macaque somatic cells was licensed by Peking University Institutional Review Board. Fibroblasts were isolated from the ear edge of rhesus monkeys and infected with retroviral vectors containing reprogramming factors OCT4 and KLF4. Medium supplemented with the small molecules was changed every 2 days. Primed iPSC colonies were selected on approximately days 25-35 after viral transduction and expanded in human ESC medium (D/F12+20% knockout serum replacements [KSR]+4 ng/ml bFGF). For naive state conversion, primed iPSC colonies were dissociated by accutase and reseeded on feeder cells. The optimized conversion medium with 4 ng/ml bFGF, 10 ng/ml human LIF, CHIR99021 (3 μM) and PD0325901 (0.5 μM), and SP600125 (10 μM) and SB203580 (10 μM) was changed daily. At approximately days 7-10, dome-shaped colonies were selected and then transferred onto fresh feeder cells for further analysis of pluripotency and differentiation characteristics.
(a) Retroviral Infection and Rhesus Monkey Primed iPSC Generation

Retroviral vectors containing reprogramming factors (OCT4, KLF4) were described in a previous report (Liu H et al., 2008). Retrovirus production, collection and infection were also conducted as described (Liu H et al., 2008 and Zhao, Y et al., 2008). Medium supplemented with the small molecules VPA (0.5 mM; Sigma), CHIR99021 (3 μM; Stemgent), 616452 (1 μM; Calbiochem) and tranylcypromine (5 μM; Tocris) was changed every 2 days. Rhesus monkey primed iPSC colonies were selected around day 25 to day 35 after viral transduction and maintained on feeder cells in the culture conditions for rhesus monkey primed iPSCs.
(b) Generation of Rhesus Monkey Naïve iPSCs from Primed iPSCs

Rhesus monkey primed iPSCs were dissociated into single cells by accutase and reseeded on feeder cells. The converting medium with 4 ng/ml bFGF (R&D Systems), 10 ng/ml human LIF (Millipore), CHIR99021 (3 μM; Stemgent) and PD0325901 (0.5 μM; Stemgent) was changed daily. At approximately day 7 to day 10, dome-shaped colonies were selected and transferred onto fresh feeder cells. Traditional 2i/LIF conditions—10 ng/ml human LIF (Millipore), CHIR99021 (3 μM; Stemgent) and PD0325901 (0.5 μM; Stemgent) were also tested and served as negative control.
(c) Conversion Condition Optimization for Rhesus Monkey Naïve iPSCs

Rhesus monkey primed iPSCs were dissociated into single cells by accutase and reseeded on feeder cells. The basal culture medium with 4 ng/ml bFGF (R&D Systems), 10 ng/ml human LIF (Millipore), CHIR99021 (3 μM; Stemgent) and PD0325901 (0.5 μM; Stemgent) was changed daily. The small molecules tested for culture condition optimization were SB203580 (10 μM; Tocris), SP600125 (10 μM; Tocris), Y27632 (10 μM; Tocris) and GÖ6983 (5 μM; Tocris). After treatment for 8 days, cells were fixed and immunostained for TRA-1-81.

Direct Conversion of Naïve iPSCs from Rhesus Monkey Fibroblasts

Retroviral vectors containing reprogramming factors (OCT4, SOX2 and KLF4) were as described (Liu H et al., 2008). Basal medium (KO-DMEM, 15% KSR) was changed every 2 days after infection. At approximately day 20, the medium was exchanged for optimized conversion medium containing 4 ng/ml bFGF (R&D Systems), 10 ng/ml human LIF (Millipore), CHIR99021 (3 μM; Stemgent), PD0325901 (0.5 μM; Stemgent), SB203580 (10 μM; Tocris) and SP600125 (10 μM; Tocris) for another 7 to 10 days. Then, dome-shaped colonies were selected and transferred onto fresh feeder cells.

### Cell Culture

Primary rhesus monkey skin fibroblasts were isolated from the ear edge of 2-year-old rhesus monkey. Fibroblasts and 293T cells were cultured in Dulbecco's modified Eagle's medium (DMEM; Hyclone) containing 10% fetal bovine serum (Invitrogen). Rhesus monkey embryonic stem (ES) cells (ES-7.5), established by Thomson et al. (Thomson J A et al., 1995), and rhesus monkey primed iPSCs were cultured and passaged as previously described (Liu H et al., 2008). Rhesus monkey naïve iPSCs were cultured in optimized conversion medium which included 85% KnockOut DMEM (KO-DMEM, Invitrogen), 15% KnockOut serum replacement (KSR, Invitrogen), N2 supplement (100×, Invitrogen), 1 mM L-Glutamine, 0.1 mM NEAA, 0.1 mM 2-ME with 4 ng/ml bFGF (R&D systems), 10 ng/ml human LIF (Millipore), CHIR99021 (3 μM; Stemgent), PD0325901 (0.5 μM; Stemgent), SB203580 (10 μM; Tocris) and SP600125 (10 μM; Tocris). Rhesus monkey naïve iPSCs were single-cell passaged every 4 days using accutase (Millipore) on feeder cells. The the TSI naïve PSC are cultured under 5% CO₂, 20% O₂, 37° C. with the optimized conversion medium and medium was changed daily.

Signaling Analysis of Rhesus Monkey Primed iPSCs and Naïve iPSCs

Rhesus monkey primed iPSCs were maintained as described (Liu H et al., 2008). Rhesus monkey naïve iPSCs were maintained in the optimized medium with 4 ng/ml bFGF (R&D Systems), 10 ng/ml human LIF (Millipore), CHIR99021 (3 μM; Stemgent), PD0325901 (0.5 μM; Stemgent), SB203580 (10 μM; Tocris) and SP600125 (10 μM; Tocris). The tested signaling modulators were SU5402 (2 μM; Tocris), SB431542 (10 μM; Tocris), Stattic (1 μM; Tocris) and TGF-β1 (2 ng/ml, Peprotech). After treatment for 5 days, cells were fixed and immunostained for TRA-1-81.

### Alkaline Phosphatase (ALP) Detection and Immunofluorescence

To detect ALP activity the cells were washed with phosphate-buffered saline three times and stained with BCIP/NBT (Promega) for 15 min. For immunofluorescence, the primary antibodies included those against SSEA-1 (1:50, Chemicon), SSEA-4 (1:20, Santa Cruz Biotechnology), TRA-1-60 (1:50, Santa Cruz Biotechnology), TRA-1-81 (1:50, Santa Cruz Biotechnology), NANOG (1:100, R&D Systems), TBX3 (1:200, Abcam), H3K27me3 (1:200, Millipore), GATA4 (1:200, Santa Cruz Biotechnology), OCT4 (1:200, Abcam) and SOX2 (1:200, Santa Cruz Biotechnology). The secondary antibodies were rhodamine-labeled donkey anti-mouse IgG (1:100, Santa Cruz Biotechnology), rhodamine-labeled donkey anti-rabbit IgG (1:100, Santa Cruz Biotechnology), rhodamine-labeled goat anti-mouse IgM (1:100, Santa Cruz Biotechnology) and rhodamine-labeled donkey anti-goat IgG (1:100, Santa Cruz Biotechnology). DAPI (Roche Applied Science) was used for nuclear staining.

### RT-PCR and Genomic PCR

Total RNA was isolated from cells using TRIzol (Invitrogen) and reverse transcribed using EasyScript Reverse Transcriptase (TransGen Biotech) according to the manufacturer's protocol. PCR amplification of different genes was performed using 2×EasyTaq SuperMix (TransGen Biotech). For genomic PCR, genomic DNA was extracted with the DNeasy

Blood & Tissue Kit (QIAGEN). The primers used are listed in Table 2.

TABLE 2
List of primers used
Primers For Quantitative RT-PCR

| | |
|---|---|
| CD44-S | CTGCCGCTTTGCAGGTGTA (SEQ ID NO: 1) |
| CD44-A | CATTGTGGGCAAGGTGCTATT (SEQ ID NO: 2) |
| DUSP10-S | ATCGGCTACGTCATCAACGTC (SEQ ID NO: 3) |
| DUSP10-A | TCATCCGAGTGTGCTTCATCA (SEQ ID NO: 4) |
| DLL1-S | GATTCTCCTGATGACCTCGCA (SEQ ID NO: 5) |
| DLL1-A | TCCGTAGTAGTGTTCGTCACA (SEQ ID NO: 6) |
| NCAM1-S | GGCATTTACAAGTGTGTGGTTAC (SEQ ID NO: 7) |
| NCAM1-A | TTGGCGCATTCTTGAACATGA (SEQ ID NO: 8) |
| SOX1-S | GGAATGGGAGGACAGGATTT (SEQ ID NO: 9) |
| SOX1-A | AACAGCCGGAGCAGAAGATA (SEQ ID NO: 10) |
| PAX6-S | AAGGATGTTGAACGGGCAGA (SEQ ID NO: 11) |
| PAX6-A | TCCGTTGGAACTGATGGAGT (SEQ ID NO: 12) |
| HPRT-S | TGACACTGGCAAAACAATGCA (SEQ ID NO: 13) |
| HPRT-A | GGTCCTTTTCACCAGCAAGCT (SEQ ID NO: 14) |
| EOMES-S | CGCCACCAAACTGAGATGAT (SEQ ID NO: 15) |
| EOMES-A | CACATTGTAGTGGGCAGTGG (SEQ ID NO: 16) |
| CDX2-S | CAGTCGCTACATCACCATCC (SEQ ID NO: 17) |
| CDX2-A | TTTCCTCTCCTTTGCTCTGC (SEQ ID NO: 18) |
| HAND1-S | AACTCAAGAAGGCGGATGG (SEQ ID NO: 19) |
| HAND1-A | CGGTGCGTCCTTTAATCCT (SEQ ID NO: 20) |
| ID1-S | AAACGTGCTGCTCTACGACA (SEQ ID NO: 21) |
| ID1-A | TAGTCGATGACGTGCTGGAG (SEQ ID NO: 22) |
| ID3-S | CTACAGCGCGTCATCGACTA (SEQ ID NO: 23) |
| ID3-A | TCGTTGGAGATGACAAGTTCC (SEQ ID NO: 24) |
| ZIC1-S | GCGCTCCGAGAATTTAAAGA (SEQ ID NO: 25) |
| ZIC1-A | GTCGCTGCTGTTAGCGAAG (SEQ ID NO: 26) |
| NANOG-S | GATTTGTGGGCCTGAAGAAA (SEQ ID NO: 27) |
| NANOG-A | CAGATCCATGGAGGAAGGAA (SEQ ID NO: 28) |
| MEXL1-S | AGCTGCTGGAGCTCGTCTT (SEQ ID NO: 29) |
| MEXL1-A | CGCCTGTTCTGGAACCATAC (SEQ ID NO: 30) |
| DNMT3A-S | AGTACGACGACGACGGCTA (SEQ ID NO: 31) |
| DNMT3A-A | CACACTCCACGCAAAAGCAC (SEQ ID NO: 32) |
| DNMT3B-S | AGGGAAGACTCGATCCTCGTC (SEQ ID NO: 33) |
| DNMT3B-A | GTGTGTAGCTTAGCAGACTGG (SEQ ID NO: 34) |
| DNMT3L-S | TGAACAAGGAAGACCTGGACG (SEQ ID NO: 35) |
| DNMT3L-A | CAGTGCCTGCTCCTTATGGCT (SEQ ID NO: 36) |
| LIFR-S | AGCGGGAGACAACACGAAAA (SEQ ID NO: 37) |
| LIFR-A | CCAGGAAGGGCATCAATCAC (SEQ ID NO: 38) |
| SOX2-S | GGCGAACCATCTCTGTGGTC (SEQ ID NO: 39) |
| SOX2-A | CAACCTGCATGGCCATTTTT (SEQ ID NO: 40) |
| ESRRB-S | TGGCTGGGTTTTGTTTGGTC (SEQ ID NO: 41) |
| ESRRB-A | TTAAAGTGTGGCCCGAGGAA (SEQ ID NO: 42) |
| ZNF521-S | CAACTGACAGATGGAGTGGATG (SEQ ID NO: 43) |
| ZNF521-A | GCTAGGGGAAGTCTGATCCTT (SEQ ID NO: 44) |
| PRDM14-S | AATCATTGGTGGCGACAACGA (SEQ ID NO: 45) |
| PRDM14-A | CCCGTACAGAACGAAGTGCAG (SEQ ID NO: 46) |
| DPPA3-S | TTAATCCAACCTACATCCCAGGG (SEQ ID NO: 47) |
| DPPA3-A | AGGGGAAACAGATTCGCTACTA (SEQ ID NO: 48) |
| TBX3-S | GAGGCTAAAGAACTTTGGGATCA (SEQ ID NO: 49) |
| TBX3-A | CATTTCGGGGTCGGCCTTA (SEQ ID NO: 50) |
| KLF5-S | CCTGGTCCAGACAAGATGTGA (SEQ ID NO: 51) |
| KLF5-A | GAACTGGTCTACGACTGAGGC (SEQ ID NO: 52) |
| REX-S | CCCTGAAGGTCATCCACAGCC (SEQ ID NO: 53) |
| REX1-A | GTGCCCATCCACATTGTCCT (SEQ ID NO: 54) |
| PRDM14-S | AATCATTGGTGGCGACAACGA (SEQ ID NO: 55) |
| PRDM14-A | CCCGTACAGAACGAAGTGCAG (SEQ ID NO: 56) |
| XIST-S | TAATGTGCCAGATACCATGCTGGG (SEQ ID NO: 57) |
| XIST-A | ACTTAACCTCACCAGTAAAGTCTTGAT (SEQ ID NO: 58) |

Primers for RT-PCR

| | |
|---|---|
| endo OCT4-S | CAGATCAGCCACATTGCCCAG (SEQ ID NO: 59) |
| endo OCT4-A | CAAAAGCCCTGGCACAAACTCT (SEQ ID NO: 60) |
| endo SOX2-S | GGTTACCTCTTCCTCCCACTCC (SEQ ID NO: 61) |
| endo SOX2-A | CCTCCCATTTCCCTCGTTTT (SEQ ID NO: 62) |
| endo c-MYC-S | GCGTCGTGGGAAGGGAGATAC (SEQ ID NO: 63) |
| endo c-MYC-A | ACCGAGTCGTAGTCGAGGTCATA (SEQ ID NO: 64) |
| endo KLF4-S | TTTTCGGTTTTGGCTTCGTTTC (SEQ ID NO: 65) |
| endo KLF4-A | GTCCAGGTCCAGGAGATCGTTG (SEQ ID NO: 66) |
| DPPA4-S | CCACCCCCGCATCTTGAA (SEQ ID NO: 67) |
| DPPA4-A | CTAACATCTGCCACCCCACC (SEQ ID NO: 68) |
| Cripto-S | CCCATGGGGATACAGCACAG (SEQ ID NO: 69) |
| Cripto-A | AAGGCAGATGCCAACTAGCA (SEQ ID NO: 70) |
| DNMT3B-S | GGTGGAGGCAGACAGTGGA (SEQ ID NO: 71) |
| DNMT3B-A | TGGTACATGGCTTTTCGATAGG (SEQ ID NO: 72) |
| SALL4-S | CGACTCGTCCTCGCTGATA (SEQ ID NO: 73) |
| SALL4-A | CCATGTTGCTTGGCCTGT (SEQ ID NO: 74) |
| NANOG-S | CCTATGCCTGTGATTTGTGGG (SEQ ID NO: 75) |
| NANOG-A | AGGTTGTTTGCCTTTGGGAC (SEQ ID NO: 76) |
| DPPA2-S | CCCCTCCCTTGCCAACCATT (SEQ ID NO: 77) |
| DPPA2-A | CACTGCCTTGCGTTTCCTCGA (SEQ ID NO: 78) |
| LIN28-S | GTTCGGCTTCCTGTCCAT (SEQ ID NO: 78) |
| LIN28-A | CACTCCCAATACAGAACACCC (SEQ ID NO: 80) |
| GAPDH-S | AATCCCATCACCATCTTCCAGGAG (SEQ ID NO: 81) |
| GAPDH-A | CACCCTGTTGCTGTAGCCAAATTC (SEQ ID NO: 82) |
| XIST-S | TAATGTGCCAGATACCATGCTGGG (SEQ ID NO: 83) |
| XIST-A | ACTTAACCTCACCAGTAAAGTCTTGAT (SEQ ID NO: 84) |

Primers for Genomic-PCR

| | |
|---|---|
| pMX-S | CCTCAAAGTAGACGGCATCGCA (SEQ ID NO: 85) |
| pMX OCT4-A | TTATTCGGGGCACCTGCTTGA (SEQ ID NO: 86) |
| pMX SOX2-A | AACCTGAGGCCCACAGTACGC (SEQ ID NO: 87) |
| pMX KLF4-A | CTCCGACAAAAGTTTCCACTCTGC (SEQ ID NO: 88) |
| pMX c-MYC-A | AGGCGTGACCGCAACGTAGG (SEQ ID NO: 89) |
| pMX GFP-A | GGGGTAGCGGCTGAAGCACT (SEQ ID NO: 90) |
| EBNA-1-S | ATCAGGGCCAAGACATAGAGATG (SEQ ID NO: 91) |
| EBNA-1-A | GCCAATGCAACTTGGACGTT (SEQ 1D NO: 92) |

### Real-Time PCR

Total RNA from an entire well of cultured cells was isolated using the RNeasy Plus Mini Kit (QIAGEN). RNA was converted to cDNA using

TransScript First-Strand cDNA Synthesis SuperMix (TransGen Biotech). PCR was conducted using Power SYBR® Green PCR Master Mix (Applied Biosystems) on an ABI Prism 7300 Sequence Detection System. The data were analyzed using the delta-delta Ct method. The primers used for real-time PCR are listed in Table 2.

### Teratoma Formation

Rhesus monkey naïve and primed iPSCs were harvested and resuspended in DF12 medium. Cells from a confluent 60-mm dish were subcutaneously injected into a non-obese diabetes/severe-combined immunodeficient (NOD/SCID) mouse (China). Teratomas formed after 6-8 weeks for rhesus monkey primed iPSCs and after 4-5 weeks for naïve iPSCs. The teratomas were then embedded in paraffin and processed for hematoxylin and eosin staining.

### Karyotype Analysis

G-band chromosomal analysis was performed at the Peking University Center of Medical Genetics.

### Mouse Embryo Micromanipulation, Whole-Mount Staining, and Imaging

For naive and primed iPSC injection, cells were trypsinized and microinjected into 8-cell stage embryos or E3.5 blastocysts of ICR diploid mouse embryo (6-10 cells per embryo). Approximately 15 injected embryos were transferred to each uterine horn of pseudopregnant females 2.5 days postcoitum. Embryos were dissected at E10-E11 developmental stages for whole-mount staining with anti-human nuclei antibody (clone 235-1, 1:200, Millipore) under the whole-mount staining procedure from Abcam. For whole-embryo sliced specimen imaging, embryos were dissected at the E16 developmental stages, followed by embedding, freezing, and slicing (10 mm thick slices), then costaining with anti-human nuclei antibody and GATA4 (1:200, Santa Cruz Biotechnology) or OCT4 (1:200, Abcam) and NANOG (1:200, R&D Systems). For confocal analysis, mounted embryos and sliced specimens were imaged by UltraVIEW VoX systems (PerkinElmer), Andor's Revolution WD spinning disk confocal microscopy system (Andor), or ImageXpress Micro High Content Screening System (MolDev).

Generation of Rhesus Monkey Naive iPSCs

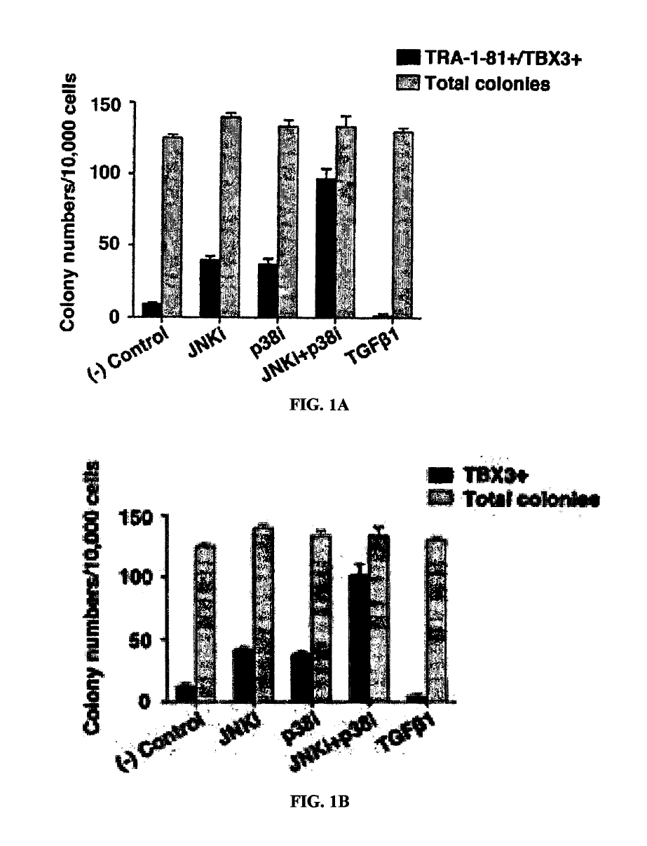

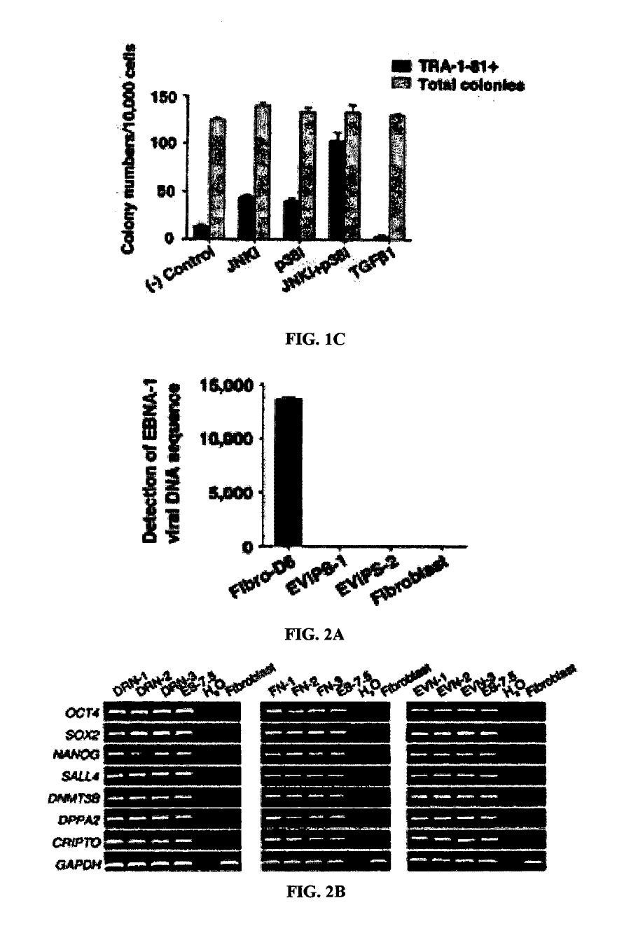

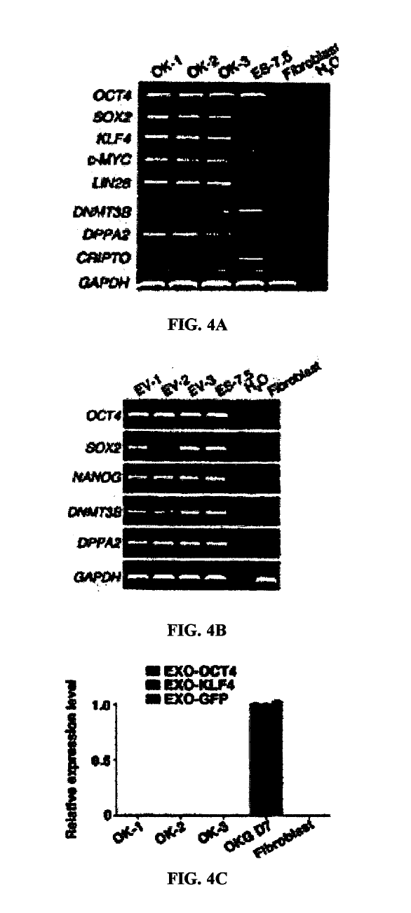

In initial experiments, rhesus monkey primed iPSC lines were first established by overexpressing OCT4 and KLF4 and culturing cells in the presence of a small molecule combination that has been reported to facilitate reprogramming with only Oct4 overexpression in the mouse (Li et al., Cell Res., 21:196-204 (2011)). Although the resulting primed iPSC lines could be maintained in human ESC medium with pluripotency in vitro and in vivo (FIG. 4A, 4C and data not shown), they rapidly differentiated and lost their pluripotency when transferred into 2i/LIF conditions that maintain naive pluripotency in mice (2i/LIF) (FIG. 1A), thus suggesting that authoritative conditions supporting rodent naive pluripotency is not sufficient for establishing the naive pluripotent state in monkey.

To convert the primed iPSCs to the naive state in the presence of 2i/LIF, several pathway modulators were tested. Dome-shaped colonies morphologically similar to mouse ESCs appeared only when bFGF, was added to the 2i/LIF conditions. Alkaline phosphatase (ALP) staining and immunostaining for the pluripotency markers TRA-1-81 and OCT4 showed that TRA-1-81-positive dome-shaped colonies were retained in the presence of bFGF after 5 days of culture in 2i/LIF conditions (data not shown). Notably, these cell colonies also express TBX3 (data not shown), a typical marker gene of naive pluripotency (Dunn et al., Science, 344:1156-1160 (2014); Niwa et al., Nature, 460:118-122 (2009)). These results indicate the importance of bFGF addition to 2i/LIF conditions within the context of converting the primed iPSCs into the naive state in monkey, although the conversion efficiency was low (8%-10% of total colonies were TRA-1-81/TBX3 double-positive and dome-shaped) (FIG. 1A and data not shown). These converted TRA-1-81/TBX3 double-positive iPSCs were identified as naive iPSCs.

Conversion Condition Optimization for Rhesus Monkey Naive iPSCs

To understand how bFGF worked to generate rhesus monkey naive iPSCs, the major pathway downstream of bFGF were investigated, the mitogen-activated protein kinase (MAPK) signaling pathway. There are at least three characterized MAPK families in mammalian cells: classical MAPK (ERK), C-Jun N-terminal kinase/stress-activated protein kinase (JNK/MAPK), and p38 kinase (Zhang, et al., Cell Res, 12:9-18 (2002)). To identify which of these had a predominant role, antagonists specific for ERK, JNK, and p38 were tested individually or in combination under the previous conversion conditions. PD0325901, an inhibitor of the classical MAPK/ERK and an indispensable component of 2i/LIF conditions, was essential for conversion and maintenance of TBX3/TRA-1-81 double-positive dome-shaped colonies (data not shown). Furthermore, two other molecules, SP600125 for JNKi and SB203580 for p38i can each greatly improved the conversion efficiency of naive PSCs in these conversion conditions (FIGS. 1A, 1B and 1C). A combination of these two inhibitors further enhanced the conversion efficiency to 75% of the total colonies (FIGS. 1A and data not shown). Moreover, when these colonies were picked and single-cell passaged, typical ALP positive, dome-shaped colonies appeared after 3 days of culture on mouse embryonic fibroblast feeder cells. Further identification by immunostaining indicated that these colonies were TRA-1-81-positive, suggesting that they remained pluripotent (data not shown).

To optimize the conversion conditions, Y27632 (a ROCK inhibitor) and GO\ddot{} 6983 (a PKC inhibitor), which have been disclosed as beneficial for the survival of and maintaining human naïve ESCs/iPSCs, were also tested (Gafni et al., Nature, 504:282-286 (2013)). However, we found that these two compounds induced pronounced differentiation and reduced TRA-1-81 expression in the colonies (data not shown), implying different requirements on signaling regulation for establishing naive pluripotency in human and monkey.

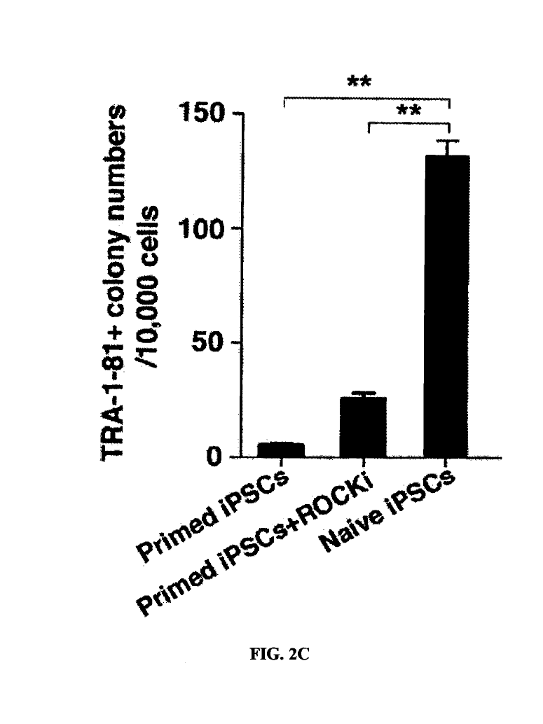

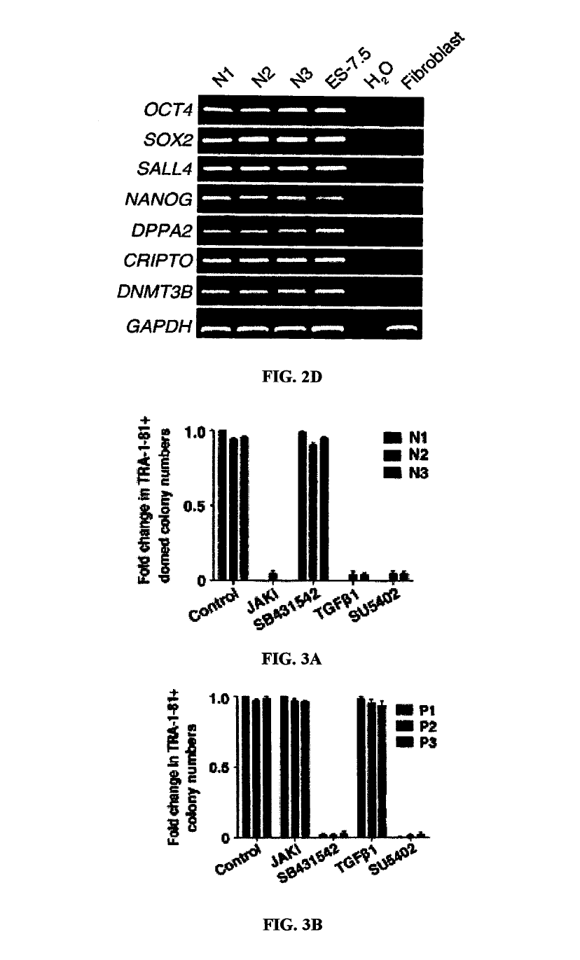

Finally, using a combination of SP600125 and SB203580 in the presence of bFGF and 2i/LIF, stable TRA-1-81/TBX3 double-positive dome-shaped naïve iPSCs were successfully established at a high efficiency (10-fold higher than 2i/hLIF+bFGF only) from primed iPSCs (FIG. 1A). Notably, with this optimized culture condition, rhesus monkey fibroblasts were reprogrammed into TRA-1-81-positive dome-shaped colonies directly by overexpression of OCT4, SOX2, and KLF4 (data not shown). This optimized culture condition was also used to establish naive iPSCs using a nonviral integration method based on episomal vectors as previously described (Okita, et al., Nat. Methods, 8:409-412 (2011)) (FIG. 2A, 2B, and data not shown) and to establish human naïve induced pluripotent stem cells (data not shown). Established naive iPSCs could be single-cell passaged every 4-5 days by using accutase and displayed high growth rates (FIG. 2C and data not shown).

Pluripotency Characteristics of Rhesus Monkey Naive iPSCs

The question of whether these naive iPSCs were indeed pluripotent was addressed using RT-PCR. RT-PCR analysis showed that naive iPSCs expressed endogenous pluripotency marker genes, including OCT4, SOX2, SALL4, and NANOG (FIGS. 2B and 2D). The pluripotent characteristics of the naive iPSCs were further examined by immunostaining. Specifically, these cells stained positive for pluripotency-specific surface markers, including TRA-1-60, TRA-1-81, and SSEA-4, but not SSEA-1 (data not shown). Additionally, naïve iPSCs showed downregulation of MIXL1, CDX2, ZIC1, HAND1, EOMES, SOX1, PAX6, DLL1, and ZNF521 (data not shown). On the other hand, these cells had normal karyotypes (42, XY for male and 42, XX for female) and maintained dome-shaped morphology and ALP activity for over 8 months of single-cell passaging (data not shown). Finally, to analyze the differentiation potential of naive iPSCs, tested their capacity to differentiate into three germ layers was. Naive iPSCs formed teratomas with tissues of all three germ layers that were detected in vivo 4-5 weeks after injection into recipient mice (data not shown). Thus, these results indicate that rhesus monkey naïve iPSCs possess pluripotent characteristics and differentiation potential.

Rhesus Monkey Naive iPSCs Possess Properties Different from Those of Primed iPSCs

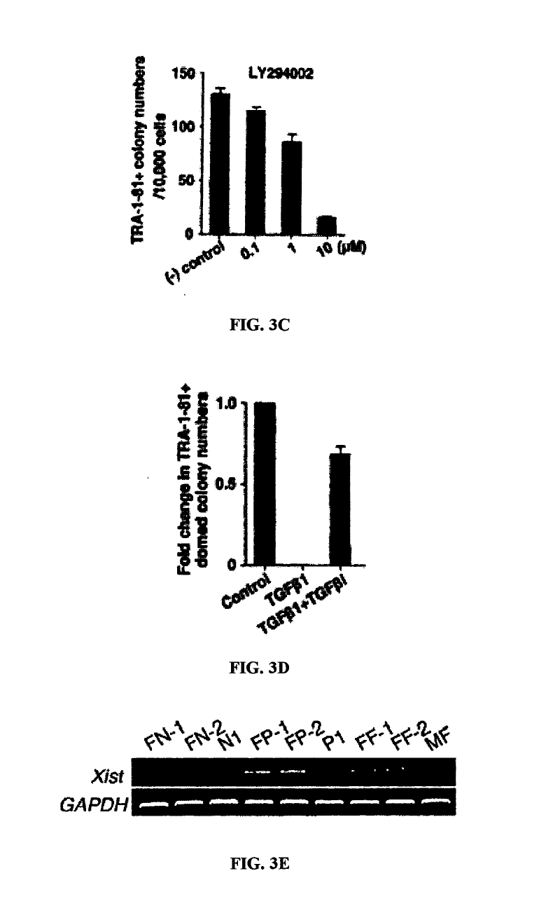

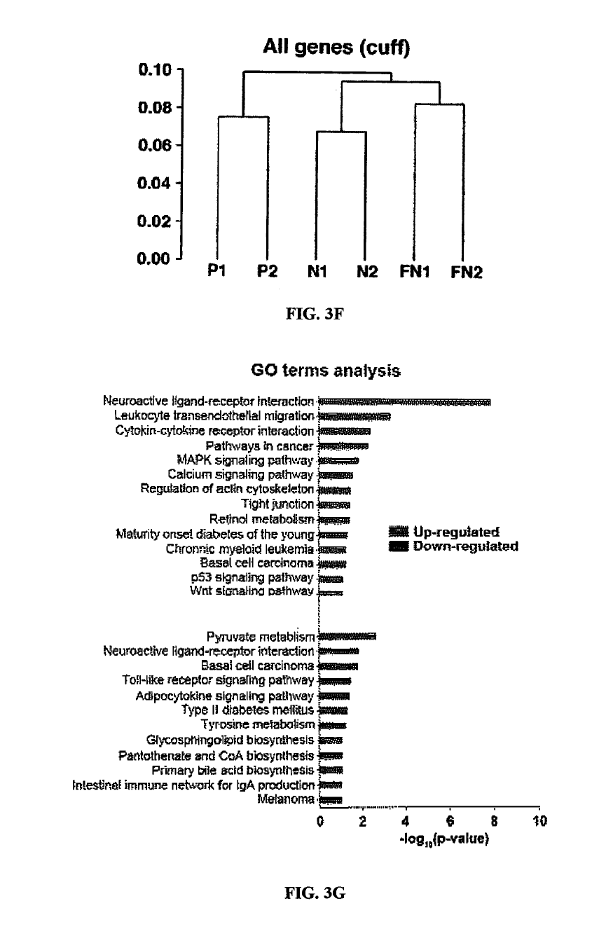

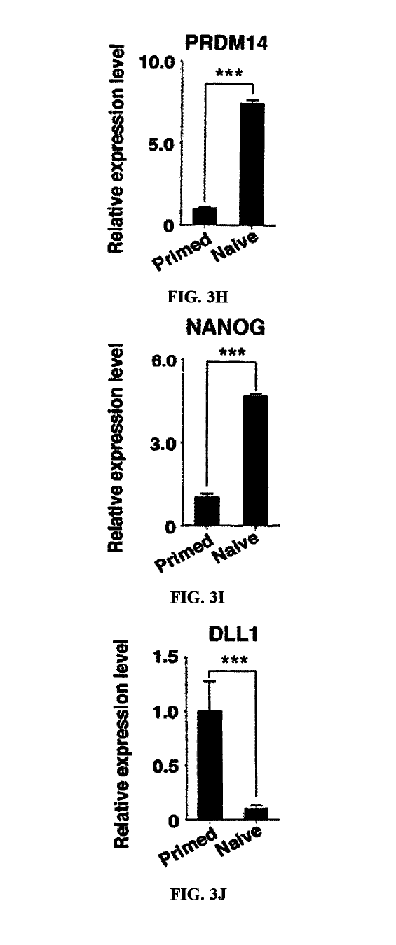

To further investigate the differences between primed and naïve iPSCs, the studies focused on the distinct response patterns of primed and naive iPSCs to different signaling stimuli or inhibitors (Greber et al., Cell Stem Cell, 6:215-226 (2010); Niwa et al., Nature, 460:118-122 (2009); Vallier et al., Dev., 136:1339-1349 (2009)). The data showed found that the self-renewal of naive iPSCs depended on the LIF signaling, similar to murine and human naive iPSCs. When exposed to a JAK/STAT3 inhibitor, naive iPSCs readily differentiated with greatly reduced expression of TRA-1-81, while primed iPSCs maintained their pluripotent properties (FIGS. 3A and 3B). Importantly, as in human cells, the bFGF signaling was required for both rhesus monkey primed and naive iPSC self-renewal. In the presence of the FGFR inhibitor SU5402, few TRA-1-81-positive colonies formed by either primed or naive iPSCs, suggesting a role for this pathway in primate pluripotency regulation (FIG. 3A and 3B). Ly294002, a typical phosphatidylinositol 3-kinase (PI3K) inhibitor, could severely block the naïve conversion process in a concentration-dependent manner, suggesting a role of FGF downstream PI3K signaling in naive state establishment (FIG. 3C). Interestingly, the addition of a specific and selective TGFIβ-RI inhibitor SB431542 to the culture medium showed no effect on rhesus monkey naive iPSCs but was devastating to primed iPSCs self-renewal, indicating that, unlike for rhesus monkey primed iPSCs and the reported human naive iPSCs (Gafni et al., Nature, 504:282-286 (2013); Theunissen et al., Cell Stem Cell, 15(4):471-87 (2014)), the TGF-β signaling was dispensable for monkey naive iPSC self-renewal. Further, The addition of TGF-β to the culture medium was devastating to the self-renewal of naïve iPSCs, but not primed iPSC (FIGS. 3A, 3B and 3D).

The X chromosome activation states in female naive iPSCs was then analyzed. Female naive iPSCs derived by the optimized condition possessed an X chromosome reactivation state, as indicated by the loss of H3K27me3 foci in the nuclei and dramatic downregulation of XIST expression levels (FIG. 3E and data not shown). In contrast, the female primed iPSCs maintain an X chromosome inactivated state, with a clear presence of H3K27me3 foci and high expression level of XIST as in somatic cells (FIG. 3E and data not shown). Accordingly, these results suggest the conservation of an X-activation status in naive PSCs among different species.

Next, the global gene expression pattern between naive and primed iPSCs was compared by RNA sequencing (RNA-seq) analysis. Genome-wide gene expression clustering showed that naive iPSCs clustered separately from primed iPSCs with a distinct gene expression pattern (FIG. 3F and data not shown). Gene ontology (GO) term analysis revealed changes of gene expression pattern related to major developmental signaling and metabolism between naive and primed iPSCs (FIG. 3G). Importantly, compared with primed iPSCs, multiple naive state-related transcripts, such as PRDM14, KLFS, ZFP42 (REX1), LIFR, TBX3, and NANOG, were upregulated in naive iPSCs (FIGS. 3F, 3G, and S3E). Meanwhile, naive iPSCs also showed a decrease in the expression of lineage-specific genes, including HOXA2, MEIS1, and DLL1, which were expressed at low but appreciable levels in primed iPSCs (FIGS. 3H, I and J). Collectively, these data indicated that the gene expression pattern of naive iPSCs was distinct from that of primed iPSCs in rhesus monkey, which is similar to previous studies in mice and humans (Gafni et al., Nature, 504:282-286 (2013); Chan et al., Cell Stem Cell, 13:663-675 (2013); Ware et al., Proc. Natl. Acad. Sci. USA, 111:4484-4489 (2014); Theunissen et al., Cell Stem Cell, 15(4):471-87 (2014)).

Finally, to test the ability of rhesus monkey naive iPSCs in generating interspecies chimeras in vivo, naive and primed iPSCs were microinjected into 8-cell stage embryos or embryonic day 3.5 (E3.5) blastocysts of ICR mice and allowed to develop to the E10-E11 developmental stages (Table 3).

TABLE 3
Summary of cross-species chimeric assay.

| Monkey Naive IPSCs Injection | | | | | | | | | Primed IPSCs Injection | | |
|---|---|---|---|---|---|---|---|---|---|---|---|
| Dev. stage | 8 cells | 8 cells | 8 cells | Blastocyst | Blastocyst | Blastocyst | Blastocyst | In total | Blastocyst | Blastocyst | In total |
| Cell line | N1 | N2 | N3 | N2 | FN-1 | DRN-1 | FN-4 | — | P1 | P2 | — |
| Injected | 49 | 61 | 32 | 50 | 104 | 20 | 21 | 337 | 47 | 57 | 104 |
| embryos | | | | | | | | | | | |
| Embryos | 11 | 21 | 14 | 32 | 60 | 12 | 10 | 160 | 23 | 27 | 50 |
| recovered | | | | | | | | | | | |
| Chimeric | 1 (E10.5) | 2 (E10.5) | 0 (E10.5) | 1 (E10.5) | 2 (E10.5) | 0 (E10.5) | 0 (E10.5) | 6 (E10.5) | 0 (E10.5) | 0 (E10.5) | 0 (E10.5) |
| embryos | 0 (E16) | 1 (E16) | 0 (E16) | 0 (E16) | 1 (E16) | 0 (E16) | 0 (E16) | 2 (E16) | 0 (E16) | 0 (E16) | 0 (E16) |

Whole-mount immunostaining of anti-human nuclei antibody (hNA), which can specifically mark the nuclei of monkey cells (data not shown), was used for detecting the presence of rhesus monkey iPSC-derived cells in vivo. Notably, in contrast to the primed iPSCs, chimeric embryos with naive iPSCs were obtained (data not shown). Particularly, three out of six chimeric embryos generated from three naive iPSC lines showed a widespread integration of naïve iPSC-derived cells (data not shown). To further investigate whether these naive iPSC-derived cells can contribute to the embryonic development of chimeric embryos, the distribution of naive iPSC-derived cells in chimeric embryos at the E16 developmental stage was analyzed. Two E16 chimeric embryos from two independent naive cell lines were further analyzed. Naive iPSC-derived cells integrated into many tissues and organs of recipient mice, such as the intestine, liver, heart, and brain. The expression of pluripotency marker including OCT4 and NANOG cannot be detected in the hNA+ cells in the chimeric embryos (data not shown), suggesting the loss of pluripotency in the naive iPSC-derived cells. Interestingly, a high-grade integration of the naive iPSC-derived cells in the heart region was also observed (data not shown). Moreover, a cardiac-specific marker GATA4 (detected by staining with a GATA4 antibody, which reacted with both human and mouse GATA4) was expressed in most of the hNA+ cells in the heart at this stage (data not shown) (Kuo et al., Genes Dev., 11:1048-1060 (1997)), implying that these naive iPSC-derived cells may further differentiate toward the cardiac fate when integrating into the developing heart. Together, these data suggest that rhesus monkey naive iPSC-derived cells could generate interspecies chimeric embryo and repopulate into the mouse early embryos with further differentiation.

### DISCUSSION

The present studies provide evidence that rhesus monkey naïve iPSCs can be successfully generated by conversion from primed iPSCs or by transcription factor-driven reprogramming of fibroblasts with a simple combination of cytokines and small-molecule inhibitors. Moreover, this conversion condition also allows long-term stable maintenance of the self-renewal circuitry of naive iPSCs. Importantly, the findings disclosed herein show that naive pluripotency, with interspecies chimeric capacity into mouse embryos, can be derived in nonhuman primates (data not shown).

The generated rhesus monkey naive iPSCs possess a number of cellular characteristics that distinguish them from primed iPSCs and conventional primate ESCs. Rhesus monkey naïve iPSCs are amenable to single-cell passaging and can be propagated for long periods with stable dome-shaped morphology and normal karyotypes. These cells respond to LIF and MAPK independent bFGF signaling to self-renew with hallmarks of pluripotency. The lack of H3K27me3 nuclear foci and downregulation of XIST transcription indicated a pre-X inactivation state of naive iPSCs. Additionally, the monkey naive iPSCs established in these study also share an expression signature with both murine and human naive pluripotent stem cells. For instance, upregulation of naive state-related genes, including NANOG and PRDM14, was observed, which serve to safeguard the murine naive pluripotent state and repress lineage commitment through the dual regulation of signaling pathways and intracellular epigenetics (Silva et al., Cell, 138:722-737 (2009); Yamaji et al., Cell Stem Cell, 12:368-382 (2013); Grabole et al., EMBO Rep., 14:629-637 (2013)). This finding is consistent with a decrease in the expression of lineage-specific genes, such as DLL1 and MEIS1 (van Es et al., Nat Cell. Biol., 14:1099-1104 (2012); Hisa et al., EMBO J., 23:450-459 (2004)), indicating a more immature state of rhesus monkey naive iPSCs (FIGS. 3H-J). Hence, the entirely distinct phenotypes of the two pluripotent states in monkey may provide an excellent model system to investigate the mechanism of the pluripotency network regulation in primates.

Another key finding in this study is that monkey naive iPSCs are capable of generating cross-species chimeras when injected into mouse embryos. Although this assay has been used to evaluate human naive PSCs, whether these cells lose their pluripotent gene expression and can further differentiate and contribute to tissues in vivo remains unclear (Gafni et al., Nature, 504:282-286 (2013); Theunissen et al., Cell Stem Cell, 15(4):471-87 (2014)). Here, whole-mount staining, whole embryo slicing, and imaging was used to obtain an overview of the distribution of rhesus monkey naive iPSC-derived cells in the chimeric monkey-mouse embryos. The costaining of anti-human nuclei antibody with tissue-specific markers and the absence of pluripotent-specific markers on the whole embryo-sliced specimen further illustrated that naive iPSC-derived cells in the chimeric embryos may further differentiate and contribute to embryo development. Accordingly, whole embryo analysis may serve an alternative strategy to provide more rigorous evidence of naive pluripotency.

These findings also indicate the evolutionary conservation and variation in the conditions for achieving the naive pluripotent state of mouse, monkey, and human cells. First, the data showed that LIF/STAT3 signaling supports the naive state not only in the monkey, but also in the other two species, suggesting a fundamental effect of LIF/STAT3 signaling in establishing the naive pluripotency network. Second, the antagonism of three central components of MAPK, ERK, MK, and p38 (Zhang and Liu, Cell Res, 12:9-18 (2002)) is important for achieving the naive pluripotent state in these species. Rhesus monkey naive iPSCs rely on bFGF signaling to sustain self-renewal. This finding is consistent with recent reports that indicated the importance and indispensability of bFGF signaling for human cells to acquire the naive pluripotent state (Gafni et al., Nature, 504:282-286 (2013); Chan et al., Cell Stem Cell, 13:663-675 (2013); Ware et al., Proc. Natl. Acad. Sci. USA, 111:4484-4489 (2014)), indicating that bFGF signaling plays a role in maintaining the naive state of both human and monkey cells. In contrast, bFGF signaling has a negative effect on the maintenance of the naive pluripotency in mouse; this could be due to the distinct genetic background of different species, as implied by the previous report (Hanna et al., Proc. Natl. Acad. Sci. USA, 107:9222-9227 (2010)). As MAPKs downstream of bFGF need to be suppressed in primate naive PSCs, other signaling pathways downstream of bFGF, such as PI3K, may exert positive effects on the naive pluripotency in primates (FIGS. 3C and data not shown). In addition, during the conversion process revealed an independence of TGF-β signaling. This is in contrast with other reports of human naive PSCs depend which on the presence of TGF-β signaling (Gafni et al., Nature, 504:282-286 (2013); Theunissen et al., Cell Stem Cell, 15(4):471-87 (2014); U.S. Publication No. 2014/0315301).

Overall, the generation of rhesus monkey naive iPSCs indicates that the two different pluripotent states (naive and primed) are conserved across species. Most importantly, the discoveries of similarities and differences among mouse, monkey, and human species in deriving naive pluripotent stem cells may aid in unraveling the mystery of the naive pluripotency and may be useful for obtaining authentic naive PSCs in other species. On the other hand, the derivation of rhesus monkey naive iPSCs also provides a valuable cell source for applications in preclinical research and disease modeling.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

1. A cell culture media composition for extending cell potency of isolated pluripotent cells, the composition comprising chemical inducer of naive pluripotency (CINP) from each of the following groups
(1) a cytokine,
(2) a glycogen synthase kinase (GSK) inhibitor,
(3 an extracellular sign regulated kinase (ERK) 1/2 inhibitor
(4) a c-Jun N-terminal kinase (JNK) inhibitor,
(5) basic fibroblast growth factor (bFGF), and
(6) a p38 mitogen-activated protein kinase (MAPK) inhibitor in amounts effective to reprogram of a non-naive cell, into a TGFP signaling independent (TSI) naive induced pluripotent stem cell (PSC).

2. The composition of claim 1, wherein the cytokine is selected from the group consisting of human inhibitory fact (LIF, “L”), interleukin (IL)-6, IL-11, IL-27, IL-31, leukemia inhibitory factor, oncostatin M, cardiotrophin-1, neuropoietin and cardiotrophin-like cytokine factor 1.

3. The composition of claim 1, wherein the GSK inhibitor is a GSK3 inhibitor.

4. The composition of claim 1, wherein the ERK) 1/2 inhibitor is selected from the group consisting of PD0325901 (N-[(2R)-2,3-Dihydroxypropoxy]-3,4-difluoro-2-[(2-fluoro-4-iodophenyl)amino]-benzamide); PD198306 (N-(Cyclopropylmethoxy)-3,4,5-trifluoro-2-[(4-iodo-2-methylphenyl)amino]-benzamide); SL 327 (α-[Amino[(4-aminophenyl)thio]methylene]-2-(trifluoromethyl)benzeneacetonitrile); and U0126 (1,4-Diamino-2,3-dicyano-1,4-bis[2-aminophenylthio]butadiene);
wherein the GSK inhibitor is selected from the group consisting of CHIR99021 [6-[[2-[[4-(2,4-Dichlorophenyl)-5-(5-methyl-1H-imidazol-2-yl)-2-pyrimidinyl]amino]ethyl]amino]-3-pyridinecarbonitrile]; BIO-acetoxime; GSK 3I inhibitor XV; SB-216763; CHIR 99021 trihydrochloride; GSK-3 Inhibitor IX [((2Z,3E)-6′-bromo-3-(hydroxyimino)-[2,3′-biindolinylidene]-2′-one]; GSK 3 IX [6-Bromoindirubin-3′-oxime]; GSK-313 Inhibitor XII [3-[[6-(3-Aminophenyl)-7H-pyrrolo[2,3-d]pyrimidin-4-yl]oxy]phenol]; GSK-3 Inhibitor XVI [6-(2-(4-(2,4-dichlorophenyl)-5-(4-methyl-1H-imidazol-2-yl)-pyrimidin-2-ylamino)ethyl-amino)-nicotinonitrile]; SB-415286 [3-[3-chloro-4-hydroxyphenyl)amino]-4-(2-nitrophenyl)-1H-pyrrole-2,5-dione]; and Bio [2′Z,3′E)-6-bromoindirubin-3′-oxime];
wherein the JNK inhibitor is selected from the group consisting of SP600125 (Anthra[1-9-cd]pyrazol-6(2H)-one); I 78D3 (4-(2,3-Dihydro-1,4-benzodioxin-6-yl)-2,4-dihydro-5-[(5-nitro-2-thiazolyl)thio]-3H-1,2,4-triazol-3-one); CEP 1347 ((9S,10R,12R)-5-16-Bis[(ethylthio)methyl]-2,3,9,10,11,12-hexahydro-10-hydroxy-9-methyl-l-oxo-9,12-epoxy-1H-diindolo[1,2,3-fg:3′,2′,1′-kl]pyrrolo[3,4-i][1,6]benzodiazocine-10-carboxylic acid methyl ester); and SU 3327 (−[(5-Nitro-2-thiazolyl)thio]-1,3,4thiadiazol-2-amine); or
wherein the p38 MAPK inhibitor is selected from the group consisting of SB 203580 hydrochloride (4-[5-(4-Fluorophenyl)-2-[4-(methylsulphonyl)phenyl]-1H-imidazol-4-yl]pyridine hydrochloride); SB202190 (4-[4-(4-Fluorophenyl)-5-(4-pyridinyl)-1H-imidazol-2-yl]phenol); DBM 1285 dihydrochloride (N-Cyclopropyl-4-[4-(4-fluorophenyl)-2-(4-piperidinyl)-5-thiazolyl]-2-pyrimidinamine dihydrochloride); SB 239063 (trans-4-[4-(4-Fluorophenyl)-5-(2-methoxy-4-pyrimidinyl)-1H-imidazol-1-yl]cyclohexanol); SKF 86002 dihydrochloride (6-(4-Fluorophenyl)-2,3-dihydro-5-(4-pyridinyl)imidazo[2,1-b]thiazole dihydrochloride).

5-7. (canceled)

8. The composition of claim 4 comprising CHIR99021; PD0325901; bFGF; SP600125; SB 203580 and SP600125.

9. (canceled)

10. The composition of claim 4 in a kit, wherein the small molecular weight compounds are present in relative amounts to put into cell culture media for differentiated cells to induce naive pluripotency.

11. A method of producing TSI naive PSC comprising:
culturing a donor cells with the composition of claim 1 for a period of time effective to reprogram a non-naive PSC into a TSI naive PSC and optionally, isolating the TSI naïve PSC.

12. The method of claim 11, wherein the donor cells are selected from the group consisting of embryonic stem cells, induced pluripotent stem cells, multipotent stem cells, cells of hematological origin, cells of embryonic origin, skin derived cells, fibroblasts, adipose cells, epithelial cells, endothelial cells, mesenchymal cells, parenchymal cells, neurological cells, and connective tissue cells.

13. The method of claim 12, wherein the donor cells are selected from the group consisting of mouse embryonic stem cells, human embryonic stem cells, and induced pluripotent stem cells.

14. The method of claim 11, wherein the donor cells are cultured for a period ranging from 4 to 14 days.

15. (canceled)

16. The method of claim 11, wherein the TSI naive PSC are seeded as single cells, the method further comprising culturing the cells in cell culture medium comprising the composition of claim 1.

17. A population of isolated TSI naive PSC obtained by the method of claim 11.

18. (canceled)

19. The population of cells of claim 17, wherein expression of at least one marker selected from the group consisting of PRDM14, KLFS, ZFP42 (REX1), LIFR, TBX3, TRA-1-60, TRA-1-81, SSEA-4 and NANOG is upregulated when compared to untreated corresponding cells isolated from the same organism as the donor cells.

20. (canceled)

21. The population of cells of claim 17, wherein expression of SSAEA-1 is down regulated when compared to untreated corresponding cells isolated from the corresponding organism as the donor cells.

22. The population of cells of claim 17 wherein the cells maintain pluripotency following culture in the presence of a TGFβ receptor inhibitor for at least five days, as measured by fold change in the proportion of TRA-1-81-positive cells relative to controls in the presence of a TGFβ receptor inhibitor.

23. The An isolated population of cells of claim 17 comprising at least 10%, 20% 30%, 35%, 40%, 45%, 50%, 55%, 60%, 65%, 70%, 75%, 80%, 85%, 90%, 95%, or 99% TSI naive PSC.

24. (canceled)

25. The cell culture of claim 1, further comprising isolated TSI naive PSC obtained by a method comprising culturing donor cells in a culture medium comprising a cytokine,
(2) a glycogen synthase kinase (GSK) inhibitor,
(3 an extracellular sign regulated kinase (ERK) 1/2 inhibitor
(4) a c-Jun N-terminal kinase (INK) inhibitor,
(5) basic fibroblast growth factor (bFGF), and
(6) a p38 mitogen-activated protein kinase (MAPK) inhibitor, wherein the culture medium effectively maintains the TSI naive PSC in an undifferentiated and naive pluripotent state for at least 2, 3, 4, 5, 6, 7, 8, 9 or 10 passages, or the TSI naive PSC maintain a normal karyotype in culture after up to 8 months in culture, or both.

26-27. (canceled)

28. A method for producing an organ in a recipient non-human mammal having an abnormality associated with a lack of development of the target organ in a development stage, comprising: a) preparing TSI naive PSC derived from a donor mammal; b) transplanting the TSI naive PSC into a blastocyst stage fertilized egg of the recipient mammal; c) developing the fertilized egg in a womb of a non-human surrogate parent mammal to obtain a litter; and d) obtaining the target organ from the litter.

29. The method of claim 28, wherein the organ to be produced is selected from the group consisting of a pancreas, a kidney, a thymus, and a hair.

30. The method of claim 28 the recipient mammal is a mouse selected from the group consisting of a Sall1 knockout mouse, a Pdx1-Hes1 transgenic mouse, a Pdx1 knockout mouse, and a nude mouse.

31. (canceled)
